(19) United States
(12) Patent Application Publication
(10) Pub. No.: US 2003/0229477 A1
(43) Pub. Date: Dec. 11, 2003

(54) Separation of matching and mapping in chemical reaction transforms

(75) Inventors: Stephan C. Schurer, Mountain View, CA (US); Steve M. Muskal, Carlsbad, CA (US); Sanjiv Kumar Jha, Sunnyvale, CA (US); Prashant Tyagi, San Jose, CA (US)

Correspondence Address: BEYER WEAVER & THOMAS LLP, P.O. BOX 778, BERKELEY, CA, 94704-0778, US

(73) Assignee: Libraria, Inc.

(21) Appl. No.: 10/373,580

(22) Filed: Feb. 24, 2003

Related U.S. Application Data
(60) Provisional application No. 60/359,643, filed on Feb. 22, 2002.

(51) Int. Cl. G06G 7/48; G06G 7/58; G06F 15/00; G01N 33/48

(52) U.S. Cl. 703/11; 703/12; 712/220; 702/19

(57) ABSTRACT

Reaction transforms described herein contain syntactically separate the matching and processing functions of a transform. Using its matching function, a transform selects specific building blocks and reacting sites from a set of building blocks. The transform selects only those building blocks and reacting sites that match a generic reactant substructure presented in the transform and possibly any other requirements and constraints present in the matching function. The transform then processes the selected building blocks by manipulating bonds and arrangements of atoms as required by the processing function specified in the transform. The processing function allows non-reaction center atoms and bonds to be processed independent from their structural context and specific chemical and physical properties. The processing function still preserves intra- and intermolecular character of a transformation.

### CROSS REFERENCE TO RELATED APPLICATIONS

The application claims priority under 35 U.S.C. §119(e) from U.S. Provisional Patent Application No. 60/359,643 (filed on February, 22 2002 by Schurer et al., and titled “Reaction Maps”), which is incorporated herein by reference in its entirety and for all purposes.

### BACKGROUND

The present invention relates to methods, apparatus and computer program products for generating and using chemical reaction transforms. These transforms allow users to generate large diverse virtual libraries from limited sets of building blocks.

The modern organic chemist has numerous software tools at her disposal. These include tools for predicting activity from chemical structure (termed “structure activity relationship” tools or SAR tools), tools for ordering commercially available reagents, and databases for storing vast quantities of chemical information including links to literature. Many of these tools have appeared recently in order to take advantage of new electronic infrastructure and electronic commerce. Others have appeared because the computational power now exists to solve previously intractable problems (or reasonably approximate a solution to these problems).

Many of these tools are intended to generate or make use of “virtual” libraries of compounds. Virtual libraries comprise representations of numerous compounds. The representations are data specifying chemical and/or physical properties of individual compounds. Typically, they identify at least the atoms and connections (bonds) that make up a chemical compound. Other properties may be included. These include bond orders, three-dimensional coordinates of atoms, charge or partial charge on atoms, stereochemical states of chiral centers, etc. Virtual libraries are usually generated with a goal of identifying compounds that have a desired activity, such as therapeutic effectiveness. To this end, the members of the virtual library may share a particular moiety, building block, synthetic pathway, pharmacophore, etc. Often a virtual library is subjected to one or more rounds of electronic screening with tools such as QSAR models, pharmacophore screens, ADME screens, and the like.

Not surprisingly, it can be difficult to generate virtual libraries whose members are broadly diverse and yet generally relevant to the project goal. This is certainly the case when virtual libraries are generated using a researcher's electronic drawing of a generic chemical reaction. It can be particularly challenging to capture all reasonable reactions within the scope of a user's generic reaction drawing. Existing software tools provide little help. They may assume certain chemical properties that are best left unspecified—particularly when these properties are used to select particular chemical building blocks (reactants) from among a set of available virtual reactants. For example, they may assume that certain the reactants are racemic mixtures, when in fact the user would like to specify a more general reaction that captures both racemic reactants and particular stereoisomers. Often, in order to capture the full range of reactants, the user will have to define multiple transformations, each for a different chemical property value (e.g., stereochemical state), to characterize a basic reaction.

What is needed therefore is an improved methodology for generating reaction transforms that can facilitate generation of virtual libraries.

### SUMMARY

The present invention fills this need by providing reaction transforms that syntactically separate the matching and processing functions of a transform. Using its matching function, a transform evaluates each specific building block from a set of building blocks to determine whether it has the reaction site specified by the matching function. The transform selects only those building blocks that match a generic reactant substructure presented in the transform that specifies the reaction site of the building block. The transform then processes the selected building blocks by manipulating bonds and arrangements of atoms as required by the processing function specified in the transform.

One aspect of the invention pertains to methods for generating a reaction transform. Such methods may be characterized by the following sequence: (a) receiving a representation of a chemical reaction for converting one or more generic reactants to one or more generic products; (b) identifying reaction center atoms in the chemical reaction; and (c) from the representation of the chemical reaction, creating a processing function for transforming representations of chemical building blocks matching the one or more generic reactants to corresponding representations of products. In this method the processing function includes (i) representations of the reaction center atoms specifying one or more of the chemical properties and (ii) representations of non-reaction center atoms, which do not include the one or more of the chemical properties. Further, the representation of the chemical reaction (i) maps atoms between the reactants and the products and (ii) specifies chemical properties for at least some atoms of the reactants and products. Generally, the non-reaction center atoms can be characterized by mapping information between the reactants and products and by connectivity information specifying their connections with the reaction center atoms. Frequently, they are characterized by an aliphatic or aromatic type, but not by a specific atom type (e.g., carbon, nitrogen, sulfur).

In many embodiments, the method creates a syntactically separate matching function from the representation of the chemical reaction. This matching function may be used for identifying building blocks having substructures of one or more reactants of the chemical reaction. It may comprise chemical properties for both reaction center atoms and non-reaction center atoms of the one more reactants. Further, it may specify a general structural or property requirement of matching building blocks (e.g., an generic aromatic group). It may also specify a conditional structural or property requirement of matching building blocks (e.g., aromatic groups, but NOT nitrogen containing aromatic groups).

In some embodiments, (a) through (c) are performed by software to automatically generate the reaction transform from the representation.

Examples of chemical properties that may be specified for atoms in the representation include stereochemical configuration, charge, isotope, oxidation state, geometric configuration, hybridization, atom mass or isotope, van der Waals surface area, electron density, electronegativity, ionization energies, Lewis basicity or acidity, and hydrophobicity.

A user may input the representation in various formats. Typically, it will comprise an electronic drawing of a chemical reaction. It such drawing does not contain the mapping, the method may automatically map an unmapped chemical reaction drawing to produce the representation of the chemical reaction received in (a).

Reaction center atoms may be identified as atoms having chemical bonds that are made, broken, or changed during the chemical reaction specified in the representation. In some embodiments, reaction center atoms also include atoms having a change in charge, oxidation state, or chirality during the chemical reaction specified in the representation.

Another aspect of the invention pertains to methods of generating a virtual library of chemical compounds from a set of virtual building blocks. Such methods may be characterized by the following sequence: (a) providing a chemical transform specifying a conversion of one or more generic reactants to one or more generic products, wherein the chemical transform comprises a matching function for matching reaction sites of building blocks and a syntactically separate processing function for mapping matched building blocks to products; (b) using the matching function to select a building block from the set of virtual building blocks and identify one or more reacting atoms in the selected building block; (c) using the processing function to modify one or more chemical properties or bond connections of the reacting atoms; and (d) generating a virtual product comprising non-reacting features from the selected building block and modified reacting atoms created at (c). Typically, the method will require repeating (b) through (d) multiple times to create a library of virtual products. Each iteration may be automatically performed by software.

Yet another aspect of the invention pertains to apparatus and computer program products including machine-readable media on which are provided program instructions and/or arrangements of data for implementing the methods and software systems described above. Frequently, the program instructions are provided as code for performing certain method operations. Data, if employed to implement features of this invention, may be provided as data structures, database tables, data objects, or other appropriate arrangements of specified information. Any of the methods or systems of this invention may be represented, in whole or in part, as such program instructions and/or data provided on machine-readable media.

These and other features of the present invention will be described in more detail below in the detailed description of the invention and in conjunction with the following figures.

### BRIEF DESCRIPTION OF THE DRAWINGS

FIG. 1A presents a schematic, high level, representation of the role of a “reaction transform” in accordance with this invention.

FIG. 1B schematically depicts how 2 transforms may be used to represent a two-step chemical process a library of virtual compounds.

FIG. 2A presents a process flow diagram depicting one method for creating a reaction transform in accordance with this invention.

FIG. 2B presents a process flow diagram depicting one method for using a reaction transform to generate a virtual library in accordance with this invention.

FIG. 3 shows a drawing of a specific chemical reaction that may be represented as a reaction transform.

FIG. 4 shows the drawing of FIG. 3 with each atom mapped between reactants and products.

FIG. 5 shows a highly generic drawing of the reaction depicted in FIGS. 3 and 4.

FIG. 6 depicts a drawing of a somewhat generic chemical reaction that falls within the scope of the reaction shown in FIG. 5.

FIG. 7 shows a drawing of an intra-molecular reaction that also falls within the scope of the generic reaction shown in FIG. 5.

FIG. 8 is a schematic depiction of a reaction transform of this invention having syntactically separate matching and processing functions.

FIG. 9 presents a loose arrangement of process operations that may be employed to generate a reaction transform of this invention having multiple matching functions.

FIG. 10 is a block diagram of a generic computer system that may be employed to implement the present invention

FIGS. 11A-11G present chemical reaction drawings for the SMARTS language chemical transforms of Examples A-G.

### DETAILED DESCRIPTION OF A PREFERRED EMBODIMENT

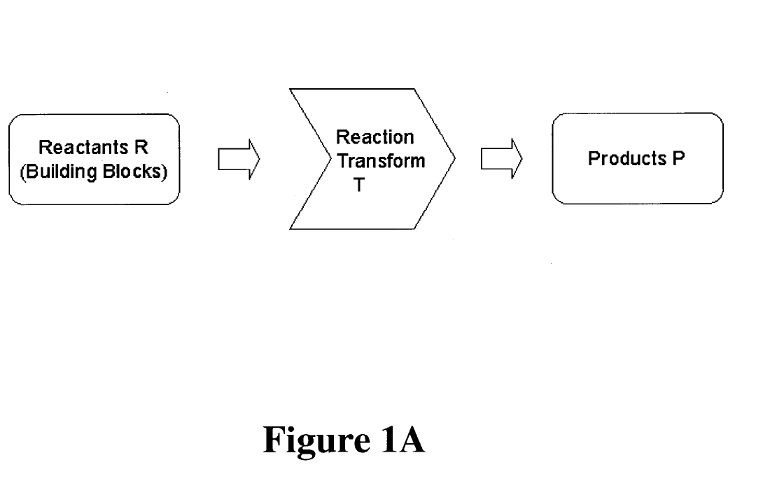

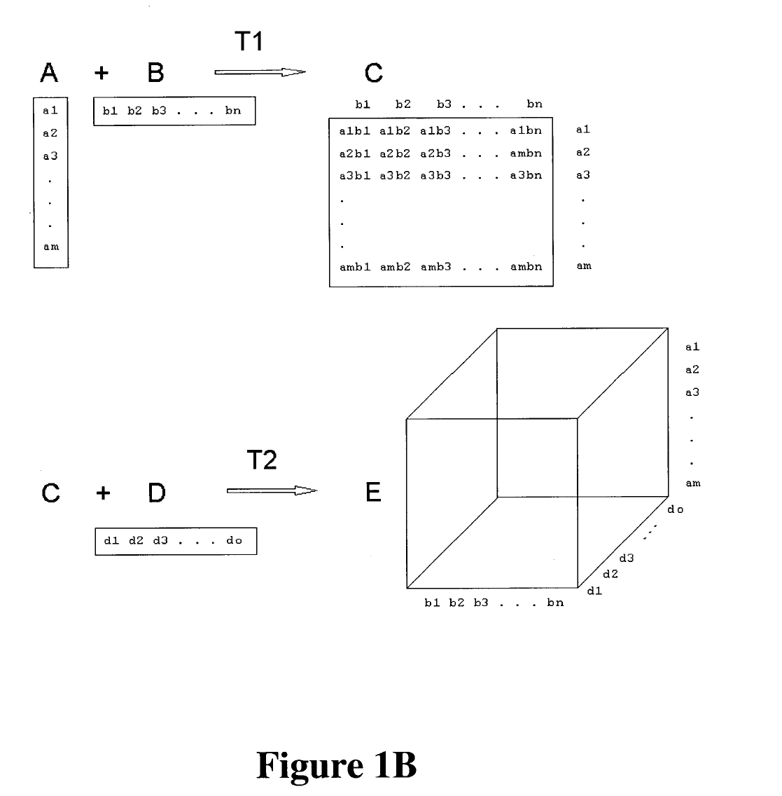

The concept of a reaction transform is central to this invention. Reaction transforms may be viewed as chemical processing instructions that are required for the computational execution of chemical reactions using suitable software or other computational resources. These transforms take one or more sets of building blocks (reactants) as input and generate products according to the processing instructions. As shown in FIG. 1A, data representing a set of reactants 101 is transformed via a chemical reaction transform 103 to produce data representing a set of products 105.

A reaction transform T performs various functions. Generally, it contains the information and instructions to analyze its input, identify the reactive atoms and bonds in each building block combination and then process these atoms and bonds—rearranging their connectivity and changing their properties according to the instructions—to exactly define the products. It should be noted that a transform, which is the focus of this application, is a function, an instruction of how to process input and generate output. Suitable software, may be required for (computational) execution of this transform. Examples of such software include the Daylight reaction toolkit, MDL Afferent, Accelrys Diversity Explorer, and generally any software that can match information on building blocks and reaction sites in a reaction-based transform and process atoms and bonds in matching building block combinations according to the information provided in this transform. Generic reaction transforms in combination with sets of building blocks are one general method to define virtual libraries. The products of the reaction transform, which are electronic descriptions of chemical compounds, comprise the virtual library.

FIG. 1B shows a two-step protocol where building blocks A (specific compounds al to am) and building blocks B (specific compounds b1 to bn) are transformed into compounds C comprising all combinations of A and B (m times n products). All members of A share a common reactive group that undergoes chemical transformation during reaction with a member of B. Likewise, all members of B share their own common reactive group that undergoes transformation when reacting with members of A. In step 2, all compounds C are then reacted with D (d1 to do) to generate the 3-dimensional virtual library E (m times n times o compounds). The two transforms used in the protocol are represented as T1 and T2. They contain the detailed processing instructions how to transform the inputs A and B to C and C and D to E respectively. In an abstracted manner, C is a function of A and B and E a function of C and D and thus A and B and D. T1 and T2 are the functions (processing instructions):

C=T1(A,B); E=T2(C,D)=T2(T1(A,B),D)

A computationally “good” transform defines the correct, expected products for each building block combination used as input. The products are usually regarded to as a virtual library. However, in the context of the complex patterns observed in synthetic chemistry, such transforms contain relatively little information. The present invention provides a method that allows researchers to enrich reaction transforms and define scope and limitations of a transform to define chemically more meaningful reaction transforms that incorporate additional information, allowing researchers to analyze structural features of a building block combination and—based on this information—process building block more selectively and chemically more meaningfully. The ideal “chemically intelligent” transform would reproduce the results that are obtained in an experiment. Obviously such a “chemical intelligent” transform is only valid in the context of reaction conditions (including temperature, time, solvent, other additives, etc).

Generally, the transforms of this invention specify syntactically separate matching functions and processing functions (sometimes referred to as mapping functions herein). Syntactic separation of matching and processing function generates more efficient transforms because a single processing function can be coupled with any matching constraints or even conditional matching constraints or requirements.

Generally, the matching function is used to select the reaction site of building blocks from a set of such building blocks. These are usually data representing specific compounds. For purposes of matching, these compounds represent potential reactants for the transform. The matching function identifies specific sites of each building block of the set that “match” the chemical constraints imposed by the transform. As an example, a matching function specifying the substructure R—C(═O)X (X meaning halogen and R allowing any substructure) would select the following building blocks: CH₃—C(═O)Cl, CH₃—C(═O)I, C₆H₅—C(═O)Cl, (NH₂)C₆H4-C(═O)Br, and CH₃C(═O)CH₂C(═O)Cl. It would not, however, select CH₃—C(═O)OH, or CH₃—C(═O)C₃H₆Cl. This matching function further includes information on which atom in the query refers to which atom in the matched building blocks. The substructure used for matching may be specified by any of a many different routes. In one typical case, it is specified by a generic reaction drawing input by a user or a SMILES representation of a reactant in that drawing. As explained below, the matching function may include additional features beyond the substructure match. These could include restrictions based on steric properties, reactivity, or stereochemical properties, for example. From the perspective of software, the matching function requires a substructure and any other matching constraints together with logic for evaluating numerous building blocks and potential different reaction sites for each building blocks to determine which of them possess the substructure and meet any additional constraints.

Note again that the matching function evaluates the entire structure of a building block to determine whether it has a reactive site as defined by the matching function. For example 1f a matching function specifies the substructure R—C(═O)OCH₃, but in addition does NOT allow R—O—C(═O)—OCH₃, this does not mean that any building block matching the prohibited substructure R—O—C(═O)—OCH₃ can be excluded. For example CH₃OC(═O)CH₂CH₂C₆H₅CH₂—O—C(═O)—OCH₃ would still be selected, but the matching function more specifically defines the reacting site of the building block.

Generally, the term “substructure” refers to a particular pattern of atoms and bonds with their properties and connectivity, leaving the context (structural environment) in which this pattern occurs undefined.

A processing function modifies reaction center atoms in a selected building block. The modification is in accordance with a transformation specified by the processing function. This transformation represents the specific type of reaction input by the user. For example, the processing function may specify a condensation reaction in which an —OH of carboxylic acid moiety is replaced with the nitrogen of a primary amine (provided from a second building block of a selected building block pair). The carbon of the carboxylic acid is a reaction center atom that is modified by the processing function to replace a —C(═O)—OH bond with a —C(═O)—N(H)— bond. In a specific embodiment, the processing function is implemented using a minimal representation of the reaction comprising reaction center atoms with all chemical properties specified and non-reaction center atoms with almost no properties specified. In one embodiment, the minimal representation preserves only connectivity and/or mapping information for non-reaction center atoms. This small amount of information allows the method to employ a single transformation to specify a broad reaction. As explained below, the connectivity information is preserved to ensure that intra-molecular reactions can be correctly represented in a single transformation.

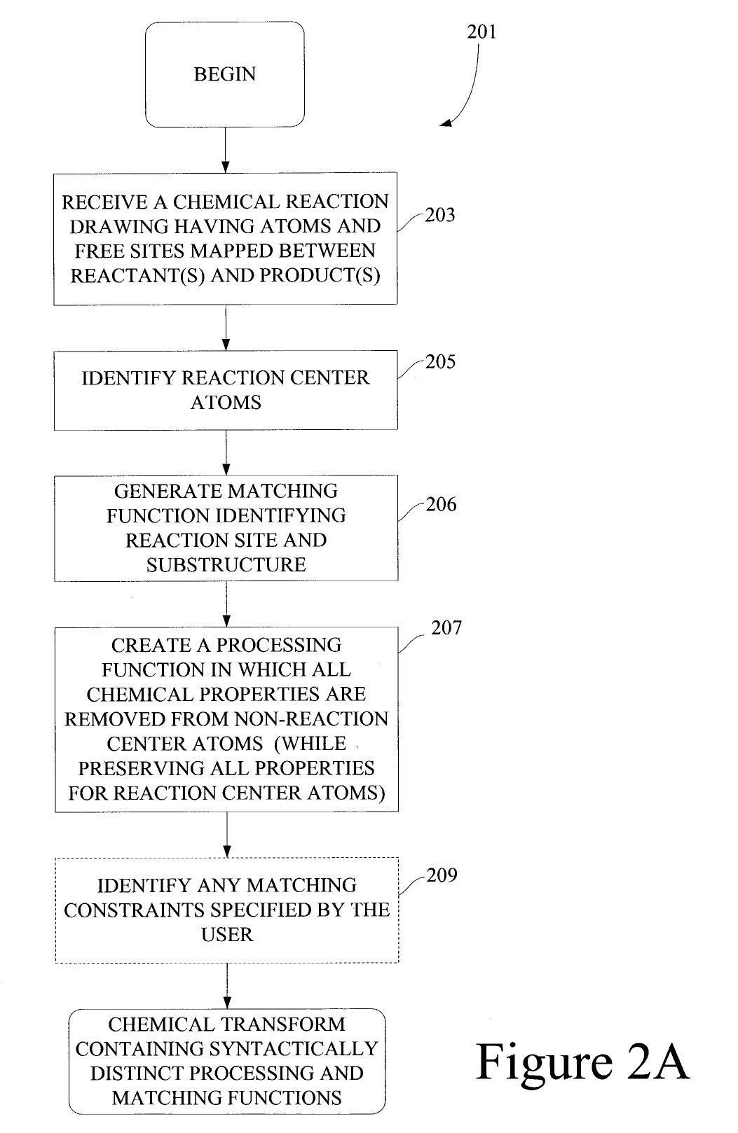

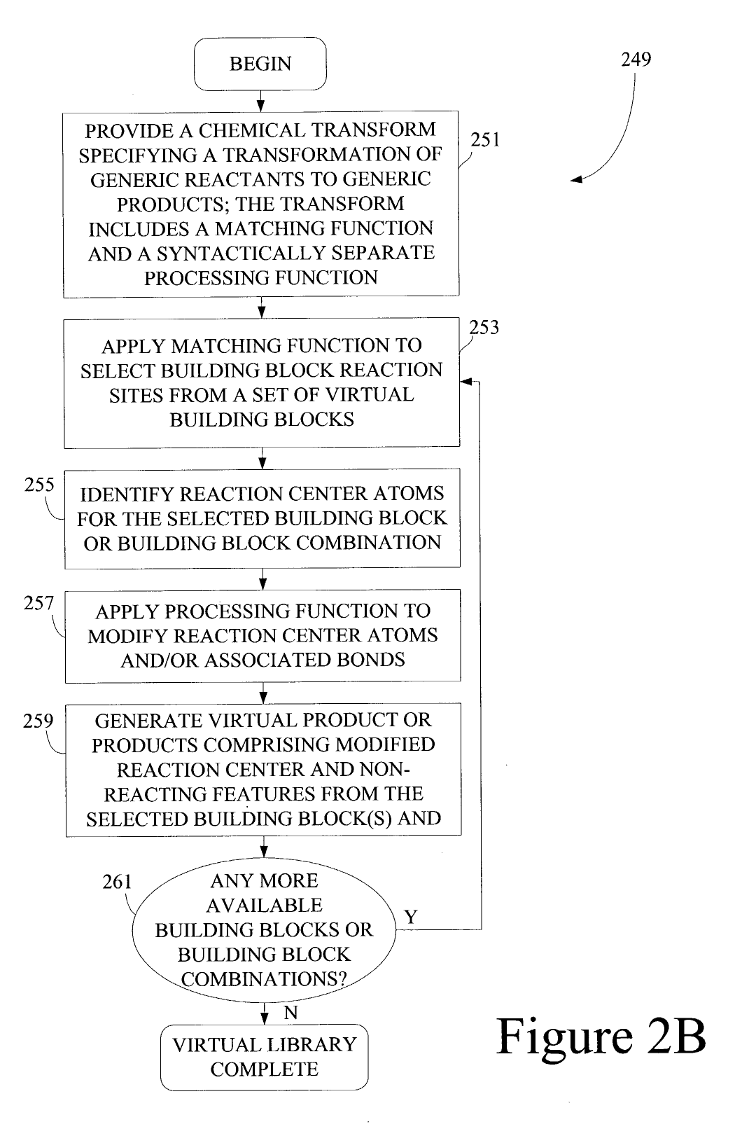

The invention will now be illustrated with reference to the process flow diagrams of FIGS. 2A and 2B. FIG. 2A presents a process that can be employed to generate a chemical reaction transform of this invention. FIG. 2B presents a process that can be employed to use a reaction transform for the purpose of generating a virtual library. Some or all of the operations shown in the flow charts can be automated. If all operations are automated in the process of FIG. 2A, the user need only supply a generic reaction drawing depicting reactants and products. The system and associated software will generate the necessary functions of the transform. For FIG. 2B, the user need only specify one or more sets of building blocks and the appropriate chemical transform (or transforms for a multi-step reaction).

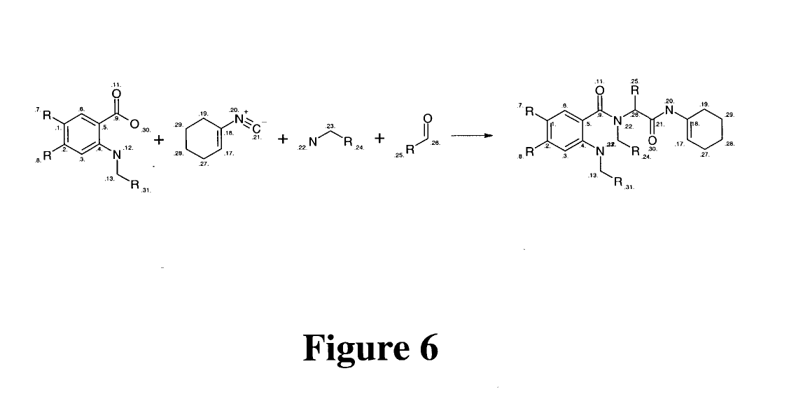

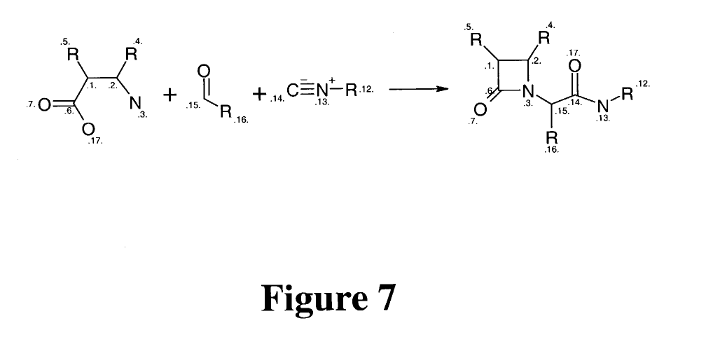

As shown, a process 201 of FIG. 2A begins at 203 with the system receiving a representation (typically a drawing) of a chemical reaction having atoms and free sites mapped between reactant and product. The “free sites” are those sites where any of many possible chemical moieties can be used. Conventionally, these are represented as “R” groups. Their use renders the reaction “generic” to some degree. Examples of reaction drawings are shown in FIGS. 6 and 7. The reaction representation is used to generate a processing function as described below. It is also employed as part of the matching function. Specifically, its reactants identify substructures used to match corresponding substructures of compounds within a building block set.

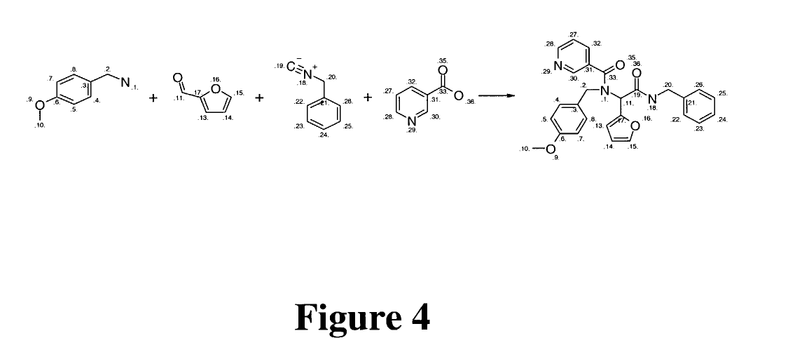

The representation/drawing may be provided in ChemDraw or MDL RXNFile format, or other convenient chemical drawing format. The representation typically depicts the individual atom types (e.g., carbon vs. nitrogen vs. oxygen, etc.). It may specify other chemical properties such as chiral orientation. The atoms of the reactant(s) and product(s) should map to one another as shown by atom numbers in FIGS. 4-7, for example. In one embodiment, the method employs a program called automapper available from InfoChem, GmbH (Germany) to auto-map RXNFiles (drawn in ISIS Draw). These can be corrected manually if they are found to be wrong.

After receiving an appropriately mapped chemical reaction drawing, the process logic next identifies reaction center atoms, as indicated at 205. The atoms of the reaction center can be identified by various techniques. Generally, the process logic determines whether an atom's connectivity or atom property changes during the transformation from reactant to product. If so, the atom is deemed to be part of the reaction center. In a specific approach, if a bond to the atom is made, broken, or changed, then the atom is deemed to be part of the reaction center. Also, if a property of the atom such as its charge, oxidation state, or chirality changes, then the atom is deemed to be part of a reaction center. Note that there can be more than one reaction center in a reactant. In the example of FIG. 7, for example, the atoms numbered 6 and 17 form one reaction center and the atom numbered 3 forms a second reaction center.

Next, at block 206, the process logic generates a matching function for each reactant identified in the chemical drawing. The matching function specifies the substructure and flags the reaction site atoms of the reactant. In one example, the matching function is a SMARTS language representation of the reactant. The reaction site atoms are listed first in the SMARTS representations.

After the reaction center atoms have been identified, the process can generate a processing function—or at least a reaction transform template for that function. See operation 207. The process logic provides only minimal chemical information from atoms and free sites that are not part of the reaction center. Frequently, the chemical representation/drawing will specify chemical properties such as oxidation state, chirality, charge, connectivity, etc. for individual atoms of the reactant or product. These may present unnecessary constraints on the range of reactions that can be handled by a single processing function. Hence, they are not included during this operation. More specifically, a processing function or template is generated that includes reaction center atoms and non-reaction center atoms. The non-reaction center atoms have few, if any, chemical properties specified, except for connectivity (which atoms are connected any given atom in the compound) and mapping information. In contrast, reaction center atoms are represented with many if not all of their properties. For example, chirality is specified. In some cases, the processing function may specify that a non-reaction center atom is simply “aliphatic” or “aromatic.” Because such limitations are so broad they need not be considered “chemical properties” in the context of this invention.

More generally, a chemical property is any chemical or physical characteristic of an atom or group of atoms in a molecule. Of relevance to this invention, at least some chemical properties are modified in reaction center atoms during transformation. In addition, chemical properties can be specified in matching functions to include or exclude reaction sites of building blocks as potential reactants. Examples of chemical properties that may be ascribed to atoms include stereochemical configuration, charge, isotope, oxidation state, geometric configuration and generally the sub-structural environment of an atom. Other examples of chemical properties are those that describe the “environment” of an atom. These include properties that require knowledge of surrounding atoms such as the orientation of an atom with respect to its neighbors and the chemical properties of its neighbors. Certain “physical” properties fall within the realm of “chemical properties” herein. These include descriptors such as hybridization, atom mass or isotope, van der Waals surface area, electron density, electronegativity, ionization energies, Lewis basicity or acidity, hydrophobicity and the like. Many of these are supported in the SMARTS and SMILES family of languages supported by Daylight Chemical Information Systems, Inc. Other languages or data formats supporting chemical properties include the MOL file format.

In summary, for those atoms found to be part of the reaction center the full representation of the atom is explicitly shown, with all relevant properties listed or annotated in an appropriate notation. For those atoms that are not found to be part of the reaction center, they are represented in a generic format. Even the identities of the atoms (e.g., carbon vs. nitrogen) are not included. The generic SMARTS notation is suitable for this purpose. In particular, a recursive SMARTS notation can be quite appropriate. Recursive SMARTS defines an atom in terms of its neighbors or neighboring groups offset by certain distance.

At this point in the process, the processing component of the reaction transform has been generated. The matching component may also have been completely generated. This depends on whether there are any matching constraints in addition to the substructure identified by the reaction drawing. If not, then the matching component is complete. In many cases, however, a user may wish to include additional matching constraints. To account for entry of these other constraints, process 201 includes an optional operation 209 for identifying additional matching constraints. These may be specified by a user or by the system logic as a default solution. In some embodiments, an appropriate user interface is provided to facilitate entry of such constraints. Note that operation 209 can be performed at nearly any stage in the process 201. For convenience, it is depicted after operation 207.

As indicated, the invention may be implemented in various chemical description languages. In some embodiments, software translates the reaction as it is provided by a chemist after mapping (mapped RXN file) into a reaction SMARTS language and then does all the operations in SMARTS/SMIRKS format; identifying reaction centers, generating matching and processing functions and then creating the final transform. In other embodiments, the software identifies the reaction center atoms in the RXN file format and then from these generates the matching and processing functions. Only after this are the matching and processing functions translated into the SMARTS/SMIRKS language.

One example a processing function in SMIRKS is shown below.

Reaction drawing:

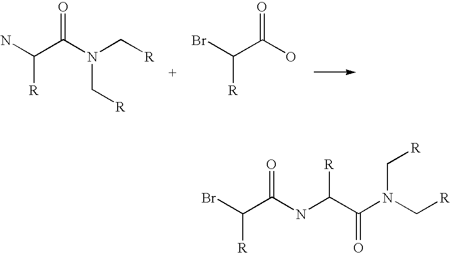

Processing function (SMIRKS):

[A:9]([A:13]([A:10][A,a:2])[A:15](=[A:8])[A:11]([[N;!H0;!H1]:5]([H])[H])[A,a:3])[A,a:1].[A:12]([A:6])([C:14](=[A:7])[OH 1])[A,a:4]>>[A:9]([A:13]([A:10][A,a:2])[A:15](=[A:8])[A:11]([N;!HO:5]([C:14](=[A:7])[A:12]([A:6])[A,a:4])[H])[A,a:3])[A,a:1]

In this example, per the SMIRKS format, the product is represented to the right of the “>>” symbol. The two reactants are separated by the “.”. In the first reactant, chemical information is specified for only the primary amine nitrogen, denoted by mapping number 5. The chemical information species that the nitrogen does not have 0 hydrogen atoms “!HO” nor does have 1 hydrogen atom “!H1.” For the other reactant, the reaction comprises a carbon atom (identified by mapping number 14) and an (unmapped) hydroxyl leaving group.

Matching components (generic reactant substructures) are illustrated, with reaction center atom appearing first. This is how the information of which atom of the matching component refers to the reaction center.

First component:

[N;!H0;!H1][C;!H0](C(═O)N([C;!H0;!H1][A,a])[C;!H0;!H1][A, a])[A,a]

Second component:

C([OH1])(═O)[C;!H0](Br)[A,a]

The complete transform (including processing and matching components) looks like:

[A:9]([A:13]([A:10][A,a:2])[A:15](=[A:8])[A:11]([A,a;$([N;!H0;!H1][C;!H0](C(═O)N([C;!HO;!H1][A,a])[C;!H0;!H1][A,a])[A,a 1):5]([H])[H])[A,a:3])[A,a:1].[A:12]([A:6])([A,a;$(C([OH1])(═O )[C;!H0](Br)[A,a]):14](=[A:7])[OH1])[A,a:4]>>[A:9]([A:13]([A:10][A,a:2])[A:15](=[A:8])[A:11]([N;!HO:5]([C:14](=[A:7])[A:12]([A:6])[A,a:4])[H])[A,a:3])[A,a:1]

The matching component for each component is inserted as recursive SMARTS in the $0 environment after the respective reaction center atoms. The reaction center atoms in the reactants of the processing functions are replaced by the generic SMARTS [A,a], because their exact properties are described in the matching function. However, they must be completely defined in the products of the processing function.

Turning now to FIG. 2B, a process 249 is depicted. This process employs a reaction transform of the type described above to generate a virtual library. The process begins at 251 by providing a chemical reaction transform of the type described above. Specifically, the transform specifies the reaction of one or more “generic” reactants to one or more “generic” products. The transform includes a matching function and a syntactically separate processing function. With such function available, the process can apply the matching and processing functions separately.

At a block 253, the process applies the matching function to select an appropriate building block (or combination of building blocks) to participate in the reaction specified by the transform. If the reaction requires two or more reactants, then a combination of building blocks must be selected, which each building block matching a different generic reactant in the transform. The building blocks are selected from a building block set, which is typically includes electronic representation of many different chemical compounds. A publicly available example of such set is the Available Chemicals_Directory from MDL (in MOL, SDF format, or SMILES format (distributed through Daylight Chemical Information Systems, Inc.)) that contains over 400.000 commercially available research grade and bulk chemicals.

The matching is performed by identifying reaction sites and building blocks having a substructure matching that of a reactant in the transform. To this end, the chemical properties and connectivities of atoms in a building block are compared with those of a reactant substructure. It may be convenient to use a consistent format such as SMARTS and SMILES for both the generic reactant in the transform and the building blocks of the set. Further, matching may be performed with appropriate software (e.g., the Daylight reaction toolkit) to match the reactant compounds against the list of building blocks provided with the user inputs.

After a building block has been selected, it may be transformed to a corresponding product. But first, the process logic identifies reaction center atoms of a selected building block or building block combination as shown at 255. These are the atoms corresponding to the reaction center atoms of the processing template reactant. By matching the building block to the substructure atom-by-atom, the process logic can easily identify the building block's reaction center atoms.

Next, at operation 257, the process logic modifies the building block's reaction center atoms and associated chemical bonds as specified by the processing function. The modification may be conducted without specifying all properties of non-reaction center atoms. If this is the case, the process generates a complete virtual product (or virtual products) by applying non-reaction center features from the original building block to the modified reaction center. See operation 259. In one embodiment, the process logic uses the SMIRKS transform function as a processing function to generate products having atoms mapped from the building blocks.

Typically, the process loops over many different building blocks available in the set. Thus, process 249 is depicted with a decision operation 261, which determines whether there are any more available building blocks or building block combinations. If so, process control returns to block 253, where the process logic applies the matching function to the current building block or combination. Thereafter operations 255, 257, and 259 are performed in succession as described above. After all available building blocks or combinations have been processed in this manner, the process is complete.

Chemical Reaction Drawings and Reaction Transforms

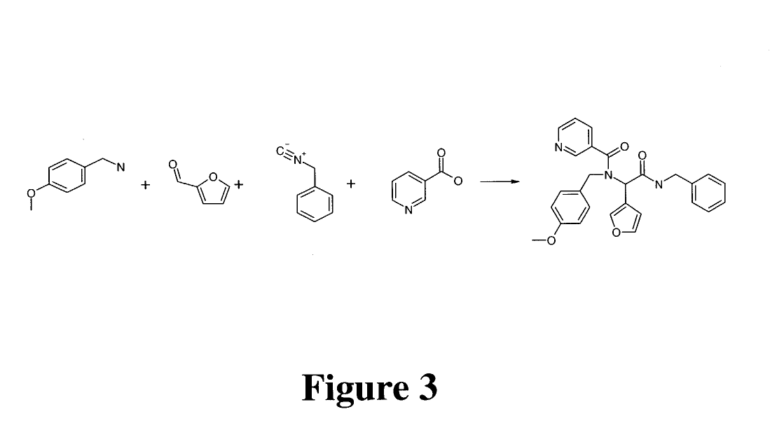

The following discussion of FIGS. 3-9 is presented to exemplify and provide a theoretical framework for specific embodiments of the invention. It also sets forth certain advantages that may be realized with this invention. However, the invention is not limited to methods and apparatus that realize these advantages.

An example for a specific reaction is shown in FIG. 3. Even though a reaction drawing such as FIG. 3 does not explicitly describe the “mechanism” of this transformation, chemists usually associate a mechanism with a reaction or a reaction type.

A common way to investigate reaction mechanisms is radioactive labeling of atoms, which allows identify which atoms in a reactant end up where in the product. A computational way to describe this is atom-atom mapping. For example FIG. 4 shows the mapped reaction of FIG. 3 reflecting the mechanism of this transformation.

The mapped specific reaction shown in FIG. 4 can be considered a chemical representation of reaction transform T that exactly describes which atoms in the reactants to translate into which atoms in the product. Any unmapped atom in the reactant does not appear in the product and any unmapped atom in the product does not have its origin in one of the reactants, but might come e.g. from the solvent.

It is important to note that in this and all subsequent representations of chemical reactions/reaction transforms, hydrogen atoms are usually not drawn explicitly. All free valences are assumed to be hydrogens and a free site is defined by an R group (see subsequent figures). This is the way chemical reactions are commonly drawn and interpreted by chemists and it is considered important to relate reaction transforms as closely as possible to the way chemists interpret a chemical reaction drawing.

Though a valid transform, a specific reaction defines only a single product from one reactant combination. The experimental validity of the specific transform in the context of specified reaction conditions (solvent, temperature, time, other additives, etc.) can easily be verified by either a reference or an experiment.

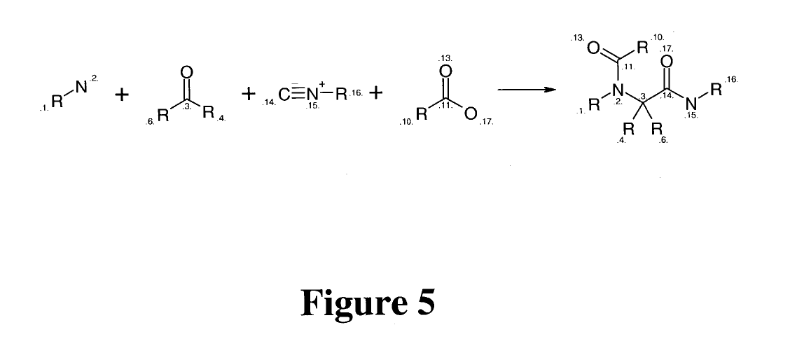

Clearly, not all atoms and bonds in FIG. 4 are directly involved in the transformation. Reaction bonds are all bonds that are formed, that are broken or that change during the transformation and, as indicated, reaction center atoms are all atoms that are connected to any such bond. For example, in FIG. 4, atoms 1,11,18,19,33,35,36 and the 0 next to atom 11 are reaction center atoms. All other atoms are herein defined as the “reaction center environment,” which usually influences a reaction indirectly. In a very generic representation, FIG. 4 could be represented as the generic reaction transform shown in FIG. 5.

In FIG. 5 all atoms except the R groups are reaction center atoms and all bonds except the ones connected to R groups are reaction bonds. Computationally R groups can be any substructure. This transform as written in FIG. 5 does not have information on the reaction center environment. Chemists drawing a reaction like FIG. 5 would usually eliminate (in their minds) some substructures of the R groups that just chemically do not make sense and thus mentally put the reaction transform in the context or a more or less general reaction center environment. For this example they would usually associate the first reactant with an amine, the second with a ketone or aldehyde, the third with an isocyanide and the forth with a carboxylic acid. Moreover, they would not consider all of these generic building blocks types to be reactive or compatible with the reaction.

The chemical representations of reaction transforms in FIG. 4 and FIG. 5, though the same reaction type, are extremes. FIG. 4 is specific and is easily experimentally verified. It is computationally not very useful, because it only contains the instructions to process a single building block combination to generate one molecule that could as well be drawn manually. FIG. 5 is very generic and it is not possible to verify all reactant combinations experimentally. It is computationally more valuable though, because it allows more building blocks combinations as input and thus—using suitable enumeration software—can generate potentially very many different products from these reactant combinations. For example the specific molecules shown as reactants in FIG. 3 could be used as input and would generate the product of FIG. 3. Many other amines, aldehydes or ketones, isocyanides, carboxylic acids can be used as input to this reaction. However there are also serious issues with a generic reaction transform as represented in FIG. 5.

(a) The definition of the output of generic transforms like the one shown in FIG. 5 is often not unique for some inputs. This happens if there is more then one match of any of the generic reactants in any of the building block combinations. This can lead to computational side reactions (computational side reaction would not occur in an real experiment, but are due to insufficient information in the transform); for example, if any R group contains a substructure that matches reactant 1, the wrong products could be defined. This problem becomes a serious issue for the computational processing of structurally more complex building blocks, where the probability of multiple matches is increased.

(b) Even if there are no computational side reactions, the transform described in FIG. 5 does not necessarily generate experimentally accessible compounds. If an R group (in any of the building blocks) contains a reactive substructure, (under given reaction conditions associated with the transform), the building block is considered chemically incompatible with this reaction. Chemical incompatibility can result in generation of side products (in an experiment), catalyst poisoning, incomplete reaction, etc. Building blocks with any such issues should not be processed.

(c) Even if there are no reactive substructures present in any of the R groups (which could lead to any of the problems mentioned above); and thus the specified reaction transform contains chemically valid processing instructions in which the R groups are not involved, their steric and electronic properties almost always have an influence on the reaction and determine whether a specific building block combination will give the desired products (as defined by the transform) in a scientific experiment or not. In other words, the reaction center environment—although not directly involved in the rearrangement of bonds and re-association of atoms—can have a significant effect on the course of a reaction and must not be ignored in a chemically meaningful transform.

The current invention provides a method that allows one to incorporate enough structural information into reaction transforms to guarantee only one valid match for each input and thus to avoid computational side reactions (problem (a) above), but at the same time remain generic enough to not exclude any input per se. The same method is used to incorporate information into reaction transforms on how R groups or generally the reaction center environment can influence the reactivity of the reaction center atoms (problem (c) above) and thus to incorporate information on scope and limitations of a reaction transform. The invention also relates to the incorporation of information on incompatible building blocks (problem (b) above), although incompatibility (with respect to given experimental conditions) can usually be identified without information of the reaction transformation (processing instructions), but just by the given substructure in the context of the experimental conditions. These various constraints may be implemented as matching function constraints as discussed above. See the discussion of block 209 in FIG. 2A, for example.

Aside from the extreme cases of the specific transform represented in FIG. 4 and the very generic transform represented in FIG. 5 other transforms, which are less generic then 5 but also less specific then 4, can be defined for the same reaction type. Two examples are given in FIGS. 6 and 7.

A person skilled in the art will readily recognize that the reaction transforms represented in FIGS. 4, 6, and 7 are all included in the more generic reaction transform in FIG. 5 (they are the same reaction type). However transforms 4, 6, and 7 contain more information on which building blocks to transform. For example, the transform in FIG. 6 requires the acid compounds to be antranilic acid derivatives, and the isocyanide to be cyclohexene isocyanide. The transform in FIG. 7 limits the amine and the acid reactant to beta amino acid derivatives. In particular it should be noted that the transform in FIG. 7 defines a (partially) intramolecular reaction, which—although it has the same reaction center atoms—is chemically very different from the intermolecular transformations in FIGS. 4 to 6.

Chemists usually draw their reactions in a more or less specific way to incorporate enough detail so suggest a final scaffold (substructure) of interest they are generating like the reactions in FIGS. 6 and 7 or suggest scope and limitations of a reaction. The specific and very general reactions in FIGS. 4 and 5 are extreme cases.

Even though transforms represented in FIGS. 6 and 7 contain more details on the reaction center environment, chemists by no means would expect any antranilic acid derivative or any beta amino acid derivative to be reactive and produce the products defined by the transform. On the other hand, for transform in FIG. 7 they might expect that the stereocenters of the alpha and beta carbon 1 and 2 of the beta-amino acid building blocks do not influence the course of the reaction and that any or some of the possible stereochemical configurations [(R),(S)], [(S),(R)], [(S),(S)], [(R),(R)], [(R,S),(R)], [(R,S),(S)], [(S),(R,S)], [(R),(R,S)], [(R,S),(R,S)] are similarly reactive. In other words, in almost all generic reactions a chemist draws, she would mentally limit or—in certain cases like for stereochemical configurations—expand the scope of the exact definition of the transform as represented in the chemical reaction drawing.

In FIGS. 4 to 7 chemical transforms are represented as chemical reaction drawings that contain substructures of reactants and products and mapping information, which reflect the mechanism of the reaction. Chemical reaction drawings created by synthetic chemists are the primary method to communicate chemical transformations. Chemists are developing, executing and reproducing chemical reactions. A reaction transform that correctly defines products from building block combinations by analyzing its input, identifying the reacting atoms and bonds in each building block combination and then processing these atoms and bonds—rearrange their connectivity and change properties according to the instructions, must relate to the reaction drawing created by the chemist and the mechanism of the transformation. Specifically it should relate to the graph (the connectivity of atoms) of the reaction drawing, even if these atoms are not reaction center atoms, because the specific connectivity of atoms can contain important additional information on the processing of a transform. For example in FIG. 7 all products are beta-lactam derivatives due to the nature of the beta-amino acid reactants. Even though carbon atoms 1 and 2 are not directly involved in the reaction (they are not reaction center atoms), they are of particular importance as they connect the two reaction center atoms N3 and C6 and thus restrict the products to be beta-lactams. They are of further importance as they are both asymmetric carbon atoms as mentioned above. However as also shown above, often the reaction drawing itself does not contain all the required information to define scope and limitations. In fact only specific reactions contain all the information. Therefore an efficient transform should reflect the chemical meaning of a generic reaction drawing, which is not necessarily exactly what is drawn.

The current invention provides general methods that provide a means to address all the issues raised above. The method enables chemically meaningful and efficient reaction transforms based on reaction drawings that incorporate the mechanism of the transformation. The transforms closely relate to how chemists draw and interpret chemical transformations. The method also allows incorporation of information defining scope and limitation of reactions independent from the graphical representation of the reaction a chemist chooses to communicate the reaction. The method is useful to define transforms that restrict the graphical connectivity of all atoms according to the reaction drawing, but at the same time leave flexibility to expand or limit the scope of the transform compared to what exact information the original reaction contains. This is particular important for reactions where a cyclization occurs as for example in FIG. 7. In FIG. 7 the intramolecular character of the transform is preserved in the processing function, thus restricting the processing to the intramolecular type defining beta-lactam products. However all other properties of non-reaction center atoms are deleted from the processing function as described above thereby making the transform more generic. In particular this allows software to process any combination of stereochemical configurations of the two non-reaction center atoms 1 and 2. The specific environment of the reaction center atoms is defined in the separate matching function, which exactly defines the beta-amino acid derivatives that can undergo the transformation. For example the allowed stereochemical configurations of atoms 1 and 2 could be restricted depending on restrictions of the other reactants of the transformation. Different configurations can be restricted depending on the environment of other reactants in the reaction; thereby exactly defining which building blocks in combination with which other ones. Notable, the processing function never has to be changed, independent from what the specific allowed/disallowed combinations of building blocks are. This closely related to what is often observed to actual organic synthetic experiments.

The concept is useful to build transforms that do not have the problems (a), (b), (c) mentioned above. Thus, the concept enables generation of valid transforms that closely relate to chemical understanding and experimental feasibility. Moreover the concept allows one to incorporate more information as it becomes available and thus to improve existing transforms with respect to scope and limitations under given reaction conditions.

Separation of Matching and Mapping Functionality in Transforms

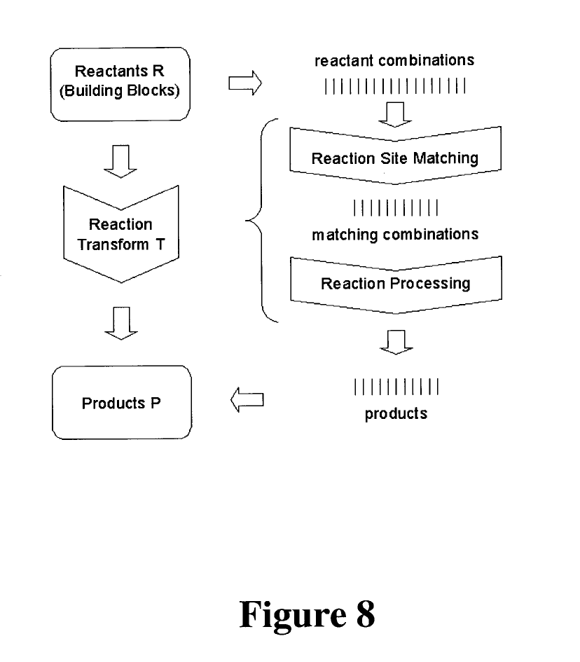

The concept that enables more meaningful chemical transforms is represented in an abstracted way in FIG. 8. FIG. 8 shows the general flow of the transformation of input reactants R into output products P. All instructions are contained in the reaction transform T as described above. As indicated, a transform contains matching information to identify the reaction sites for each building block combination and the actual processing instructions for how to rearrange and change properties of atoms and bonds to define the product output. A chemical reaction that contains atom-atom mapping information represents a transform T. It contains both matching and mapping (processing) information. Matching and mapping form an integral part of the transformation and are usually not separated syntactically in a chemical reaction. The atoms maps are directly associated with the reactant (and product) atoms, which form a substructure, which functions as the mapping component (see FIGS. 4 to 7).

Representation of the separation of matching and mapping (processing) information as shown in FIG. 8 is not necessarily meant to be a temporal separation, but rather a syntactic separation. Mapping and matching functionality are (syntactically) separated in the notation of the transforms. In an abstracted way the transform T can be considered a combination of two functions M and P for matching and processing (mapping). So if the final products C as in FIG. 1B are defined as C=T1 (A,B); C can be defined C=P1[M1(A, B)]. Similarly products E can be represented as

E=T2(C,D)=T2[[P1(M1(A,B))],D]=P2{M2[[P1(M1(A,B))],D]}

In all cases M and P are syntactically separated in the notation of the transform.

The separation of matching and processing function M and P in a transform T has some general advantages.

The matching functionality does not have to be the same substructure as the atom-atom processing instructions (the mapping information). Additional information can be incorporated in the matching functionality to define scope and limitations of the transformation. It should be noted that the matching functionality does not select the building blocks that are allowed to enter a transform, but rather selects the specific reaction sites, the reaction center atoms and bonds to be processed. Required or prohibited environments of the reaction center atoms can be defined by adding more matching functionality. Moreover conditional and logical dependencies among different reactants can be defined. So the matching function M can actually comprises multiple components M1, M2, M3, . . . Mn, which incrementally define the scope and limitation of the transform. The information represented by M1, M2, . . . Mn can generally be incorporated from any possible source, for example expert knowledge, other negative or positive references of the same reaction type, experimental results, etc. Again entry of such matching functions is depicted at block 209 in FIG. 2A. A suitable language to describe matching functions is the Daylight SMARTS language.

Separation of the matching functions M from the processing function P in a transform T also allows one to use the same processing function independent from how complex the matching function is. This is achieved by reducing the processing instructions (how to change atom connectivity and bonds and what atoms and bond properties to change) to only the actual reaction center atoms and bonds. This does not make the reaction more generic, which could define additional unintended products, because the information is preserved in the matching function M. Limiting the processing information to the reaction center atoms does not mean that all non-reaction center atoms are deleted from the mapping function. It rather means that the definition of how atoms connectivity and bonds and associated properties change is limited to reaction center atoms. Mapping instructions for all other atoms and thus their connectivity is retained in the reaction transform. This is required for the efficient processing of transformations with an intramolecular cyclization as mentioned above for the example in FIG. 7. However the related structural environment and any properties, in particular stereochemical configurations, of these non-reaction-center atoms and bonds are deleted from the P function. Any non-reaction-center atoms and bonds thus are processed independent from their structural environment and their properties, because these do not change during the reaction. The structural environment and all properties are preserved in the separated matching function M.

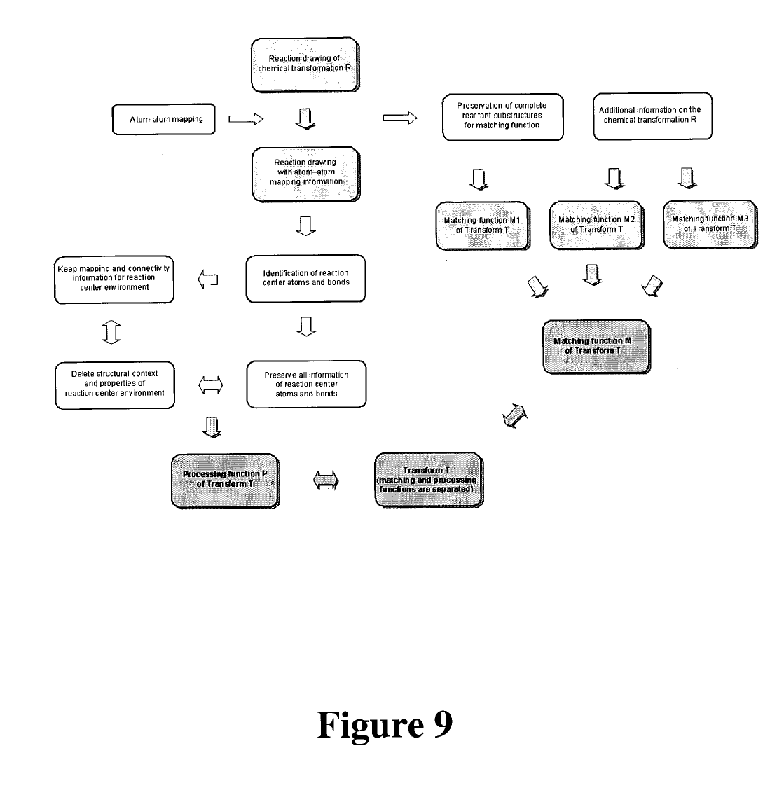

A loose schematic depiction of how one can generate transforms with separated matching and processing functions from chemical reaction drawings is given in FIG. 9. This figure overlaps substantially with FIG. 2A.

The process starts from a chemical reaction drawing created by a chemist, e.g. FIGS. 3 to 7, except that chemists usually need not explicitly include mapping information. After atom-atom mapping, a mapped reaction drawing in a standard reaction file format (e.g., a RXN file format) is generated. Mapping information can be generated by suitable software e (e.g. the automapper by Infochem GmbH, Germany) or manually or a combination thereof. As mentioned above, the mapping information reflects the reaction mechanism.

From the original reaction drawing in combination with mapping information, the complete substructure of each generic reactant is preserved as a separate entity. Mapping information is not required in this substructure, but it is important that the information which atoms/bonds in this substructure refer to which reaction center atoms/bonds is preserved. The generated substructure(s) with this information provides the matching function M1. Additional information (from different sources as mentioned above) on scope and limitations of the transformation R can be translated into other matching functions M2, M3, etc. All matching functions M1, M2, M3, etc. may be logically combined to define the matching function M, which contains all information on the environment of the reaction center atoms and bonds that was fed into the functions and thus allows to identify only reactive combinations of reaction centers in the building blocks (or if no reactive atoms are identified to exclude certain building blocks completely).

From the mapped reaction transformation the reaction center atoms and bonds are identified. This information can be obtained by analyzing the difference in connectivity and properties of each atom and bond in reactants and products.

To generate the processing function, all the information in the original mapped reaction is preserved for reaction center atoms and bonds. For non-reaction center atoms and bonds all information (structural context and properties) except the connectivity of the atoms and the mapping information is deleted. The result is a processing function with reduced matching information—a more generic transformation then the original mapped transform R. Only original information of how to process the reaction center atoms and bonds are preserved and thus restrictions that might be in the reaction center environment of the transformation, in particular those of a stereochemical nature, are eliminated. All non-reaction center atoms and bonds are still preserved as points and nodes of connectivity and the mapping information ensures that all atoms and bonds are translated correctly into products. These non-reaction center atoms and bonds are just decoupled from their environment and their properties and are translated into products independent from their properties and environment. In a way the shape of these atoms and bonds, the connectivity and the mapping information, is maintained, but the specific environment is deleted. One may therefore speak of shape-based transforms in this context. To generate useful transforms, the reaction center environment should not be deleted completely in the processing function, but the connectivity of the atoms and the mapping information should be kept, despite the additional separately kept matching function. The two main reasons for this are as follows.

(a) To base the processing instructions on the original drawing of the transformation R, which chemists use to communicate chemical reactions (among each other, but also for example via a user interface and suitable software with a computer), and thus to facilitate automation of the generation of these processing functions from reaction drawings using suitable software and a user interface and

(b) to generate valid transforms for all intramolecular reactions, where two or more reaction center atoms in one reactant are connected by non-reaction center atoms as for example in FIG. 7. Preserving connectivity and mapping information of reaction center environment maintains the intramolecular character of the transformation in the processing instructions. The driving force of intramolecular and intermolecular reactions are fundamentally different and it is believed that in a chemically meaningful transform this fundamental difference cannot be ignored. However, deleting the structural context of all non-reaction center atoms and bonds and all their properties, generates the most generic processing instructions in the context of the character and the type of the transformation. For example the stereochemical context (environment) of reactants can be translated into the products without defining the stereochemical information itself.

To generate the final transform T that can be processed by software, the matching instructions M are linked to the reaction center atoms and bonds of the processing instructions P. Specifically, in one embodiment, the matching function for each reaction center in each reactant is inserted as a recursive SMARTS after the respective reaction center atoms in the processing function. Thus, scope and limitations of a transformation can be incorporated in the transform independent from the source of the information. The processing instructions are generalized, but the shape of the reaction center environment is preserved in these processing instructions, allowing for example to differentiate intra- and intermolecular reactions.

For example, if an intramolecular transformation of the type in FIG. 7 only works with certain stereochemical configurations or certain stereochemcial configurations only work in combination with certain building blocks of another reactant, this information can be incorporated in the matching function M of the transform and thus will exclude unreactive building blocks or specific unreactive building block combinations. The processing instructions P are generalized to allow all stereochemical information—independent from the actual configuration—to be translated correctly into the products, because all structural context is deleted from these atoms. Thus, only one transform is required for all stereochemical transformations, which results in a significantely more efficient transform. The intramolecular character of this reaction is also preserved, because the connectivity information of the two chiral non-reaction center atoms and bonds is maintained in the processing instructions.

One specific application of the above described concept of syntactic separation of the matching function M and processing function P in a transform T is the definition of generic stereochemistry in a transform. This was briefly mentioned above in the context of FIG. 7 and is described in more detail in Example 1 below:

Hardware/Software Implementation

As should be apparent, embodiments of the present invention employ processes acting under control of instructions and/or data stored in or transferred through one or more computer systems. Embodiments of the present invention also relate to apparatus for performing these operations. Such apparatus may be specially designed and/or constructed for the required purposes, or it may be a general-purpose computer selectively activated or reconfigured by a computer program and/or data structure stored in the computer. The processes presented herein are not inherently related to any particular computer or other apparatus. In particular, various general-purpose machines may be used with programs written in accordance with the teachings herein. In some cases, however, it may be more convenient to construct a specialized apparatus to perform the required method operations. A particular structure for a variety of these machines will appear from the description given below.

In addition, embodiments of the present invention relate to computer readable media or computer program products that include program instructions and/or data (including data structures) for performing various computer-implemented operations. Examples of computer-readable media include, but are not limited to, magnetic media such as hard disks, floppy disks, magnetic tape; optical media such as CD-ROM devices and holographic devices; magneto-optical media; semiconductor memory devices, and hardware devices that are specially configured to store and perform program instructions, such as read-only memory devices (ROM) and random access memory (RAM), and sometimes application-specific integrated circuits (ASICs), programmable logic devices (PLDs) and signal transmission media for delivering computer-readable instructions, such as local area networks, wide area networks, and the Internet. The data and program instructions of this invention may also be embodied on a carrier wave or other transport medium (e.g., optical lines, electrical lines, and/or airwaves).

Examples of program instructions include both low-level code, such as produced by a compiler, and files containing higher level code that may be executed by the computer using an interpreter. Further, the program instructions include machine code, source code and any other code that directly or indirectly controls operation of a computing machine in accordance with this invention. The code may specify input, output, calculations, conditionals, branches, iterative loops, etc.

The various method operations described above may be implemented in whole or in part on one or more computer systems. Often, it will be convenient to employ two or more nodes to implement the invention. As an example, one node may serve to provide a program for generating a virtual library of chemical compounds, while a different node provides a database or database management system for accessing data representing building blocks to be matched and transformed by the program. Such nodes may share information via a computer network. In addition, separate computer program products (including their machine readable media) may be employed to house the program instructions for separate operations of the invention.

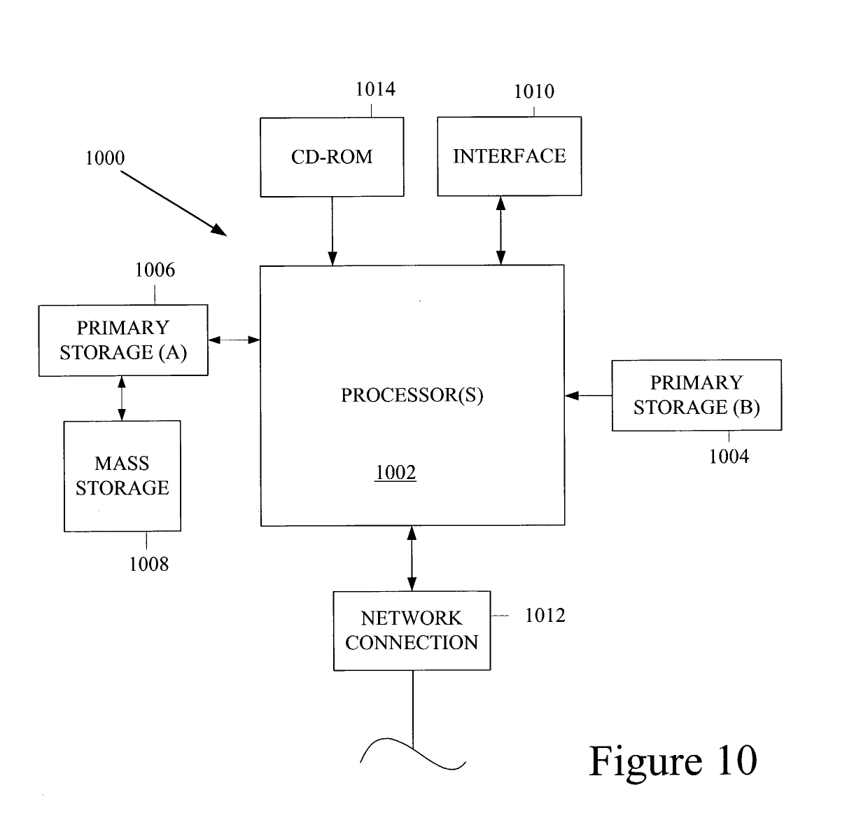

FIG. 10 illustrates, in simple block format, a typical computer system that, when appropriately configured or designed, can serve as a computational apparatus of this invention. The computer system 1000 includes any number of processors 1002 (also referred to as central processing units, or CPUs) that are coupled to storage devices including primary storage 1006 (typically a random access memory, or RAM), primary storage 1004 (typically a read only memory, or ROM). CPU 1002 may be of various types including microcontrollers and microprocessors such as programmable devices (e.g., CPLDs and FPGAs) and unprogrammable devices such as gate array ASICs or general purpose microprocessors. In the depicted embodiment, primary storage 1004 acts to transfer data and instructions uni-directionally to the CPU and primary storage 1006 is used typically to transfer data and instructions in a bi-directional manner. Both of these primary storage devices may include any suitable computer-readable media such as those described above. A mass storage device 1008 is also coupled bi-directionally to primary storage 1006 and provides additional data storage capacity and may include any of the computer-readable media described above. Mass storage device 1008 may be used to store programs, data and the like and is typically a secondary storage medium such as a hard disk. Frequently, such programs, data and the like are temporarily copied to primary memory 1006 for execution on CPU 1002. It will be appreciated that the information retained within the mass storage device 1008, may, in appropriate cases, be incorporated in standard fashion as part of primary storage 1004. A specific mass storage device such as a CD-ROM 1014 may also pass data uni-directionally to the CPU or primary storage.

CPU 1002 is also coupled to an interface 1010 that connects to one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 1002 optionally may be coupled to an external device such as a database or a computer or telecommunications network using an external connection as shown generally at 1012. With such a connection, it is contemplated that the CPU might receive information from the network, or might output information to the network in the course of performing the method steps described herein.

In one embodiment, the CPU of computer system 1000 is configured with logic (e.g., software, firmware, etc.) to provide a multidimensional browser and/or image alignment tool as described herein. In one example, system 1000 corresponds to the client machine 101 depicted in FIG. 1. The objects used by the browser may derive from various sources including mass storage device 1008 or an external database or file server such as entities 119 and 117 shown in FIG. 1. Other data such as the filter parameters and corresponding filter values may be found likewise be stored in such locations. Remote sources of the objects or other data may provide their information to the information to system 1000 via interface 1012.

Once in the apparatus 1000, a memory device such as primary storage 1006 temporarily stores the information. The memory may also store various routines and/or programs (temporarily or persistently) for analyzing and presenting the data. Such programs/routines may include database management systems, search engines, programs for mapping, programs for identifying reaction centers, programs for generating chemical transforms, programs for matching building blocks to reaction transforms, programs for transforming selected building blocks, programs for populating databases with virtual libraries, tools for improving the performance of databases, etc.

### EXAMPLE 1

### Addressing Stereochemistry with Reaction Transforms

General

A problem in the accurate description of generic reactions is the incorporation of the stereochemical environment of the reacting atoms. In cases where the configuration of the stereocenter does not influence the cause of the reaction like a peptide coupling reaction (scheme 1) a generic description of stereocenters should be found; and the specific configuration of a stereocenter in a reactant should be transformed into the product without change—according to the nature of the chemical transformation. Thus, if the configuration of the R1 building block in scheme 1 is (S) it must be (S) in the product, if it is (R), the product must have (R)-configuration at this position and if it is not defined (racemic) also the product must be racemic at this carbon (see table 1).

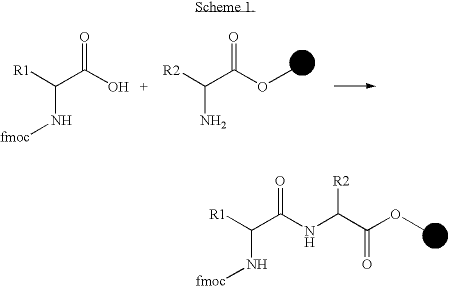

One could argue that this chemical reaction could also be described by the more generic amide bond formation in scheme 2, having the stereocenters hidden in the R groups.

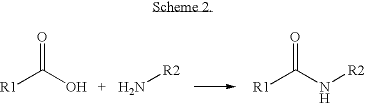

However organic chemists usually describe their Markush reactions as complete as possible, including more than just the reacting functional groups in order to show the specific product type synthesized and also to suggest scope and limitations of a synthetic methodology. This is of particular importance for multi-step reaction sequences, where a product of one reaction step becomes a reactant of the next step. By showing more complete generic molecules, the reaction sequence and the synthesized product type become apparent from the graphical description. The specific structures of building blocks are not required to understand the reaction sequence. In the majority of cases organic chemists therefore use the least generic representation of a Markush reaction to communicate synthetic chemistry. In some cases, a more generic representation does not exist. A common example is an intramolecular reaction, where the connection between two reacting functionalities must be shown. FIG. 7 above should serve as an illustrative example.

Stereocenters are often present in reactant molecules. If one required the specific definition of all stereocenters in a generic reactant (outside the reacting functional groups) the number of reactions necessary to describe all possible stereochemical combinations would be 3ⁿ with n the number of stereocenters in all reactants. Therefore a description of undefined (not R, not S, not racemic) stereocenters in the reaction center environment would be useful. The specific configuration of these stereocenters is determined by the specific building blocks incorporated in the chemical transformation assuming present chiral centers do not influence the cause of the chemical reaction, the reaction rate is independent from the specific stereoisomeric configurations, and the stereocenters do not change during the reaction (table 1). In a case where a stereocenter changes its configuration it becomes a reaction center and has to be specified to describe the nature of the chemical reaction.

For the communication of generic reactions that fulfill these criteria a generic representation according to scheme 1 could be defined.

TABLE 1

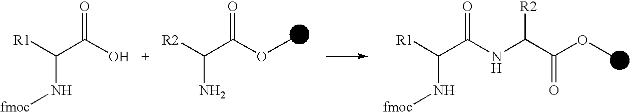

| Reactant 1 | Reactant 2 | Product |
|---|---|---|
| 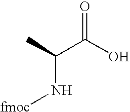 | 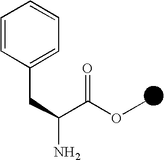 | 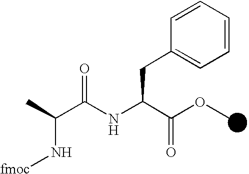 |
| 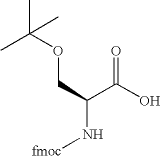 | 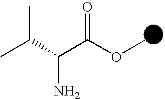 | 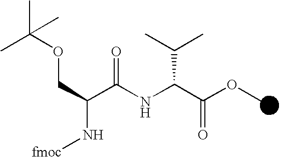 |
| 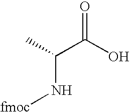 | 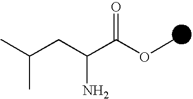 | 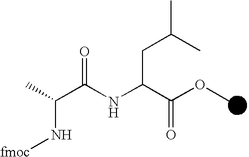 |
| 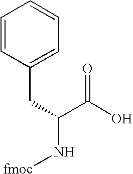 | 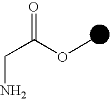 | 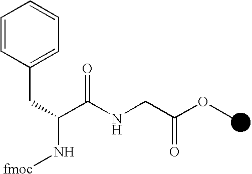 |

Computational Requirements/Implications:

The above considerations become particularly important when a Markush reaction has to be translated into a function that can be processed by a computer to accomplish a transform-based enumeration process.

It is important to note that in these cases the translation into a transform is not just a simple 1:1 translation, but rather a translation of the meaning of a chemical reaction drawn by a synthetic chemist and not a computational chemist. For example the acylation of a support-bound amine with an amino acid could be described as in scheme 3 in case of natural amino acids. In this case an unnatural amino acid would react under similar reaction conditions, but is not included in the generic reaction in scheme 3, because it would have the opposite configuration. Also a racemic amino acid would not match the reaction in scheme 3. Therefore the meaning of the reaction would better be translated with a generic (undefined) stereocenter. The specific stereocenter for each example is defined in the building blocks.

If the reaction is computationally processed, the specific stereocenter from each reactant has to be transferred into the product as described above with examples in Table 1.

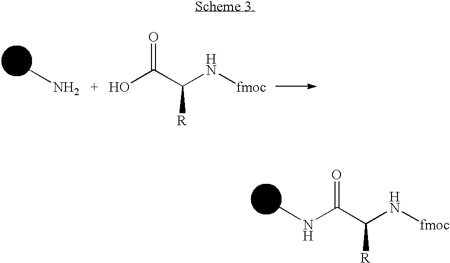

Results

The following describes a method to translate chemical reactions into chemically meaningful transforms. In the processing functions, stereocenters that do not influence the cause of a chemical reaction are not defined (not R, not S, not racemic). The transform functions transfer the stereochemical information from the reactant building blocks into the products. This results in the enumeration of products with the correct stereochemistry (as it would be expected chemically) without the necessity to define the stereochemistry in the processing function itself.

Thus, the method allows the direct translation of complex multi-step reaction sequences including all details in the reaction center environment—in particular for chiral building blocks—into a chemically meaningful function. This function can be processed very efficiently, because only one processing function is required for each reaction step of the sequence and this function is valid for all stereochemical combinations outside the reaction center.

Reaction Transforms:

As indicated above, a transform generally comprises a description of the reactant molecules that matches the specific reactant molecules to the generic reactant in the transform (a matching function) and a mapping function (processing function) that maps atoms on the product site of a chemical reaction with atoms on the reactant site. Thus each chemical reaction can be considered a (specific) transform if the reactant and product atoms are mapped—numbered—and the same atoms in the reactants and products have the same mapping number. Reactions containing free sites—represented as R groups—are simple generic transforms (for example scheme 4).

Such transforms can be described using standard file formats like the MDL RXNfile or the Daylight SMIRKS language. The Daylight SMIRKS language has been designed to allow the enumeration of product molecules using the Daylight reaction toolkit and SMILES reactant molecules as input.

As can be seen in scheme 4, a simple transform would require a separate reaction (processing function) for each stereochemical combination. In the reaction drawn, the transform would only allow unnatural (R configuration) amino acids in solution and natural (S configuration) amino acids on support. If the stereocenters are deleted, all products are defined racemic (per SMARTS interpretation) and the transform would racemize the stereocenters of the reactant molecules. This would result in an invalid transform because in this specific example chemically no racemization occurs.

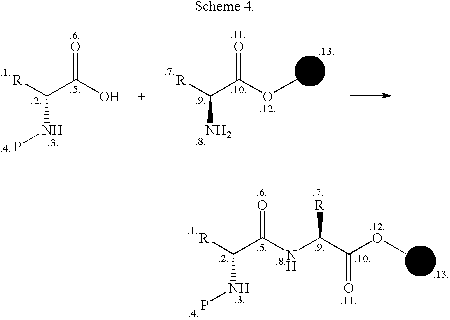

For the reasons mentioned above, an undefined (not R, not S, not racemic) stereochemical description for reactant molecules is required for an efficient processing transform function.

General Process of Generation of Transforms with Undefined Stereochemistry:

As explained above, the problem is solved by syntactically separating the matching function of a reaction transform from the mapping function. Since an undefined stereochemistry can only be applied for non-reaction center atoms the method first separates the reaction center atoms from the non reaction center atoms. As indicated, a reaction center atom is typically defined as an atom involved in making, braking or changing a bond to any other atom or an atom that changes any other property (such as oxidation state/charge, configuration/chirality etc.). Based on this definition, reaction center atoms can be identified in standard file formats such as RXNfiles or SMIRKS. For any other (non-reaction center) atoms other chemical properties are deleted from the transform, except the atom mapping function and the connectivity of the reactant and product atoms—defined in an RXNfile as a connection table or in a SMIRKS by the linear sequence of atoms. Specifically the stereochemical information is deleted from the reactant and product molecules of the transform. To create a chemically meaningful transform it is important to delete only the stereochemical information that does not influence the cause of the reaction. For this purpose reactions can be classified into categories where each stereocenter can be analyzed for its importance for the reaction (see below for an example how reactions involving stereocenters could be categorized). In the MDL language, stereochemistry is described by relative coordinates, which can be directly deleted to create a generalized transform with undefined stereochemistry. In the Daylight SMIRKS language chirality is an atom property in combination with the atom sequence in the SMIRKS file. It can be deleted by replacing each non-reaction center atom by a generic SMARTS that matches all atoms with all properties. This could be visualized as in scheme 5, where only the 2-dimensional shape (of the reaction in scheme 4) of a molecule and the mapping function of the reaction are retained, except for reaction center atoms where all properties must be defined in the reactants and products.

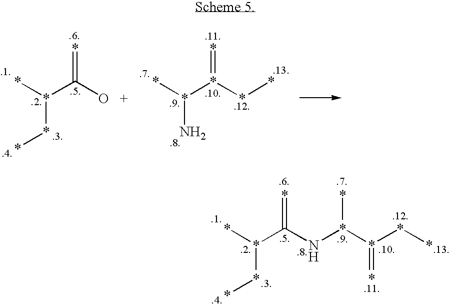

The reactant molecules of the generalized transform now match all stereochemical configurations, but also other undesired reactants. Therefore a separate matching function is incorporated into the transform, which specifically defines the reactant molecules. This can be done by insertion of a recursive SMARTS component after one reaction center atom of the generalized transform. If the recursive component is inserted after a non-reaction center atom, wrong products can be observed due to undesired mismatches in rare cases.

The recursive component simply is generated from the initial (non-generalized) reaction. The transform created in the described way matches all reactants independent from the specific configuration of stereocenters that do not influence the chemical reaction, but no other undesired reactants. Due to the generalized description of all non-reaction center atoms in the reactants and the products in case of the Daylight SMIRKS, all atom properties—including the specific stereochemical configurations—are carried from the reactant molecules to the product molecules during the enumeration, because the mapping function of the generalized atoms is maintained.

In case of transforms in the MDL RXNfile format, the stereochemical information (defined by relative coordinates) that is not relevant for the reaction can be deleted directly. The specific configuration (relative coordinates) of each reactant molecule is directly carried to the product molecules.

A C-program has been written that generates such transforms (with syntactically separate matching and processing function) in the Daylight SMIRKS language from mapped RXNfiles, which are created by organic chemists with a drawing package like ISIS draw or ChemDraw.

As indicated above, Daylight Chemical Information Systems, Inc. has designed various notations for languages for representing organic compounds and reactions involving such compounds. The basic notation for entering and representing molecules is known as “SMILES.” It is a widely used line notation. A related Daylight language is “SMARTS.” SMARTS is similar to SMILES, except that it additionally allows for generic substructure representations, as opposed to explicit atom-by-atom designations. Specifically, the SMARTS language allows one to set atoms as wildcards, generic aromatic atoms, generic aliphatic atoms, etc. It also allows specification of certain environment or property characteristics or specific atoms. Examples include valence, ring size, connectivity, charge, ring membership, chirality, etc. Also, SMARTS allows substructures to be represented with logical operators such as AND, OR, and NOT.

SMILES and SMARTS can also be used in reaction transform notations that specify reactants and products associated with a given reaction (transform). The related language, known as SMIRKS, is defined for generic reactions. It requires a one-to-one mapping between atoms specified in the reactants and atoms specified in the products (however, no mass-balance is required). The atoms may be represented explicitly (in SMILES format) and/or generically (in SMARTS format). Typically, reactions are represented in SMIRKS/SMARTS format (SMIRKS are reaction SMARTS).

A generic SMIRKS/SMARTS representation of a reaction transformation allows a chemist to identify a wide range of potential products that can be produced from various combinations of reactants available to the chemist. The Daylight reaction toolkit can be used to enumerate the molecules using a SMIRKS (as reaction processing instructions) and SMILES as reactant molecules as input and generating SMILES as product molecules. To this end, a matching function (e.g., a SMARTS representation of the reactants) is used to match available reactants (building blocks) against reactants designated in the transform function. From these selected building blocks, the processing function then maps the reactant atoms to a product.

Again, while the description herein focuses primarily on the SMIRKS/SMARTS/SMILES family of Daylight languages, the principles of this invention apply to various other “languages” or notations for representing chemical compounds and reactions.

As explained in this example, sometimes chemists enter reactions graphically, showing chiral orientations. Other times they do not. Either way, the chemist is often interested in identifying all reactions (or products thereof) that can be produced from a certain set of building blocks (reactants) available to her. Some of these available building blocks may be stereoisomers of one another. Others may be racemic mixtures of the isomers. For many reactions, the chemist wishes to identify all possible reaction products producible from two or more chiral building blocks that each may be available in R form, S form and/or racemic form.

Unfortunately, most conventional reaction notations (such as SMIRKS) make assumptions about the stereochemistry of the product based upon the form of the input SMILES or SMARTS description. These notations allow one to specify chirality as an atom property for a chiral center. For example, in SMILES notation, a chiral center annotated with the “@” symbol indicates that the three succeeding neighbors in the representation are oriented anti-clockwise. The symbols “@@” indicates that the succeeding neighbors are oriented clockwise. If no “@” symbols are specified for the chiral center, the Daylight software assumes that the product compound is racemic. In other words, racemic products are generated if a stereocenter in a product is not specified.

To address this issue, atoms found to be part of the reaction center or otherwise important in the reaction are fully represented, with all relevant properties listed in the notation. But those atoms that are not found to be part of the reaction center (or of a moiety that influences the reaction significantly) are represented in a generic non-chiral format. The generic SMARTS notation is ideal for this purpose. In particular, a recursive SMARTS notation can be quite appropriate. Recursive SMARTS defines an atom only in terms of its neighbors or neighboring groups offset by certain distance. No assumption about chirality is made.

### EXAMPLE 2

### Stereochemical Classification of Chemical Transformations Involving Stereocenters

One protocol for determining how to generate processing functions for various classes of stereochemical reactions is set forth below. To generate chemically meaningful transforms in this approach, only stereocenters that do not influence the chemical reaction should be undefined.

Specific examples are now given for various classes of stereochemical reaction.

1.) Reaction Center is not Chiral

1.1) One chiral center in reaction center environment (atoms outside the reaction site); e.g., an amide formation under non-racemizing conditions:

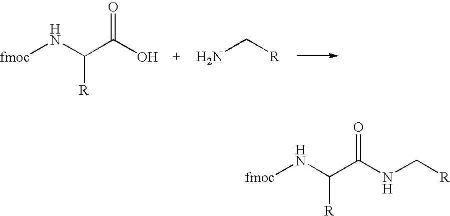

1.2) More than one chiral center in environment

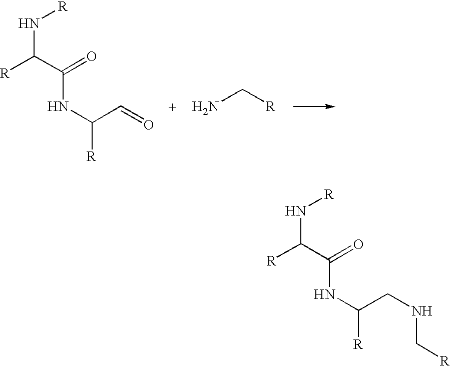

Stereochemistry cannot be undefined if different diastereomers react in considerably different rates. In combinatorial chemistry generally different diastereomers are considered equally reactive.

2) Reaction Center Itself is Chiral or a Chiral Center if Formed in the Reaction

The stereochemistry of the reaction center can never be undefined if the reaction is not racemic.

2.1) Reactant center achiral, product center chiral

Formation of stereocenter depends on reaction center environment (diastereoselective reaction); formation is relative to an existing stereocenter. In this case the stereocenter in the reactant (that determines the new product stereocenter) and the stereochemistry in the product has to be specified. For each enantiomer a separate transform is required. Consider the following reaction.

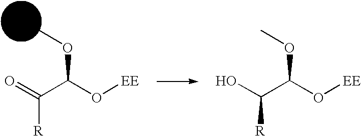

Another common example is the Evans Aldol reaction.

2.1.2) Formation of stereocenter is independent from reaction center environment; e.g., a chiral catalyst is employed. The formation is absolute; e.g., the chiral Ru-catalyzed Noyori reduction below yields the shown product independent from the configuration of the —OAc group.

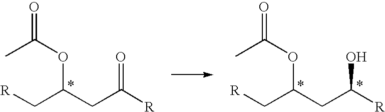

If the formation of the new stereocenter does not depend on existing stereocenters in the environment, these should be undefined to distinguish this case (absolute formation of stereocenter) from the diastereoselective reaction (relative formation of stereocenter).

2.2) Reactant center chiral, product center achiral

The racemizing stereocenter has to be specified in the reactant. For each enantiomer a separate transform is required.

E.g. the racemization of an N-acyl protected amino acid via azlacton formation:

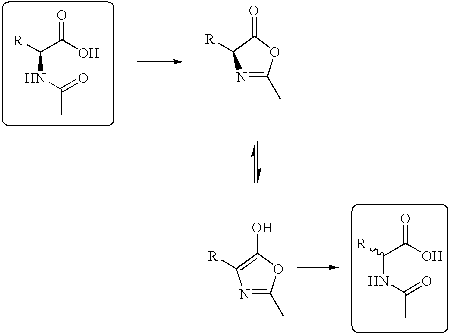

2.3) Reactant and product centers are chiral

In most of the cases stereocenters in the environment of the reaction center do not influence the cause of the reaction (inversion vs. retention) and therefore can be undefined.

2.3.1) Inversion of configuration

E.g. the cyclative S_{N}2 thioalkylation

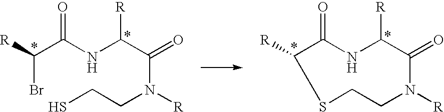

For each enantiomer a separate transform is required.

2.3.2) Retention of configuration. Similar to 2.3.1)

For each enantiomer a separate transform is required.

### EXAMPLE 3

### Specific Transforms in SMARTS

Each example shows the (unmapped) reaction drawing as provided by a chemist; the mapped reaction transformation (atom-atom maps can be generated automatically or manually or a combination thereof) and the transform function with syntactically separate matching and processing functions. The atom mapping numbers and the order of the reactants and products in the drawings and the transform functions are not the same. Also the processing function and the matching components are given separately. In the final transform function, the matching components are linked to the processing function as a recursive SMARTS, which is inserted in parentheses after the “$” symbol.

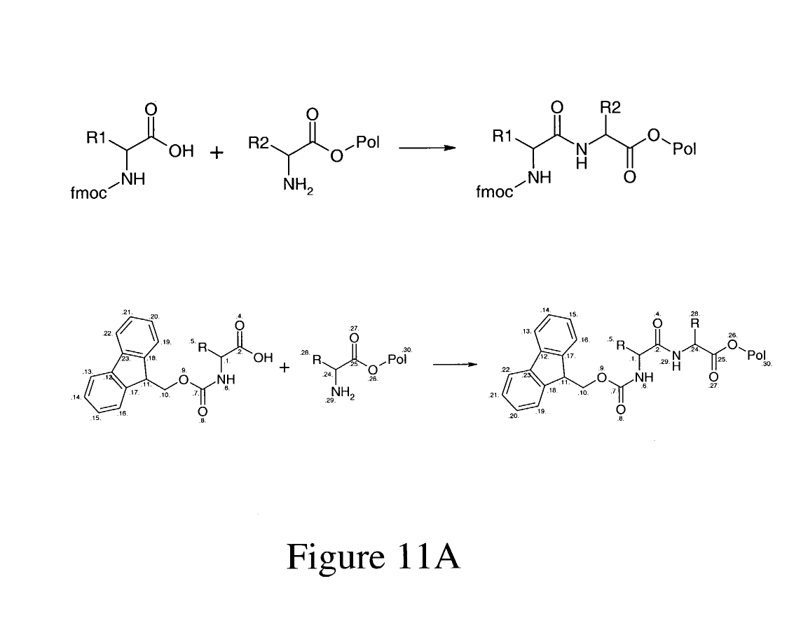

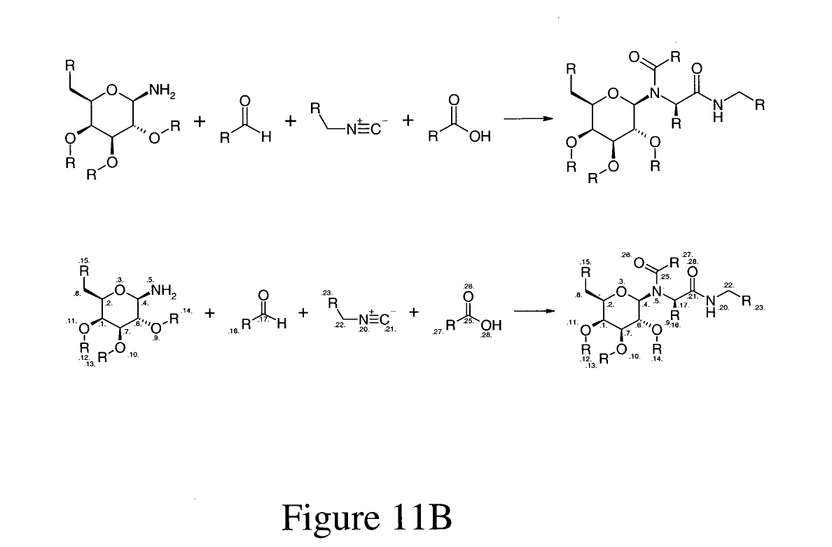

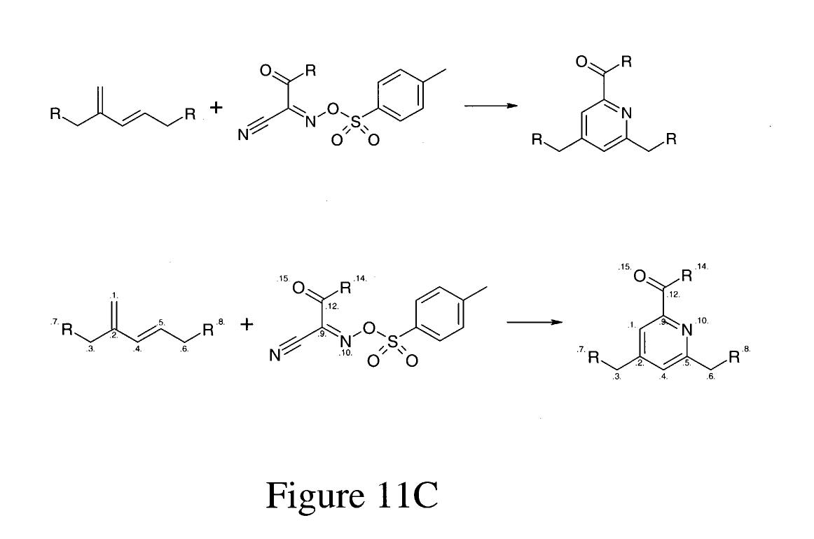

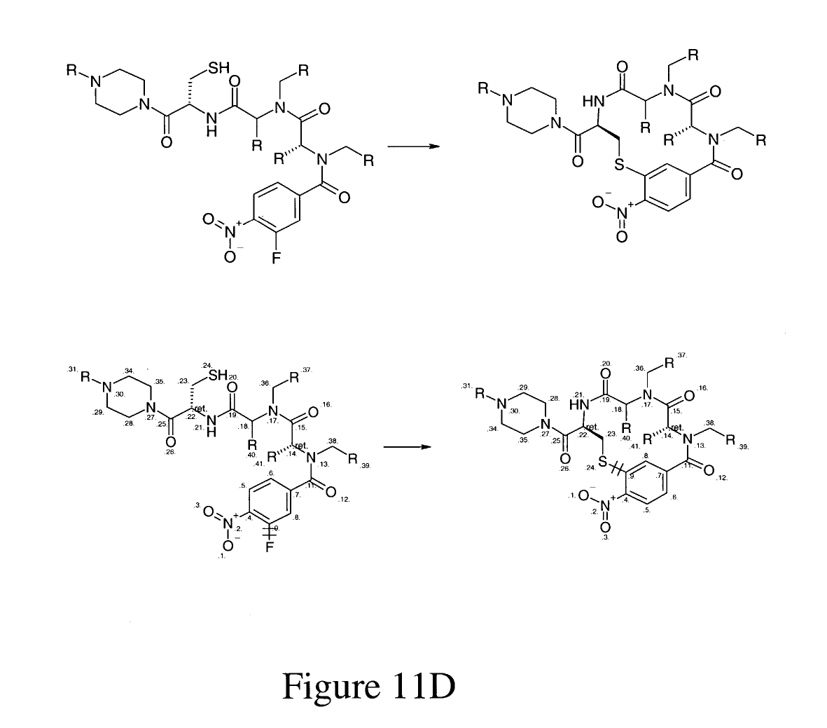

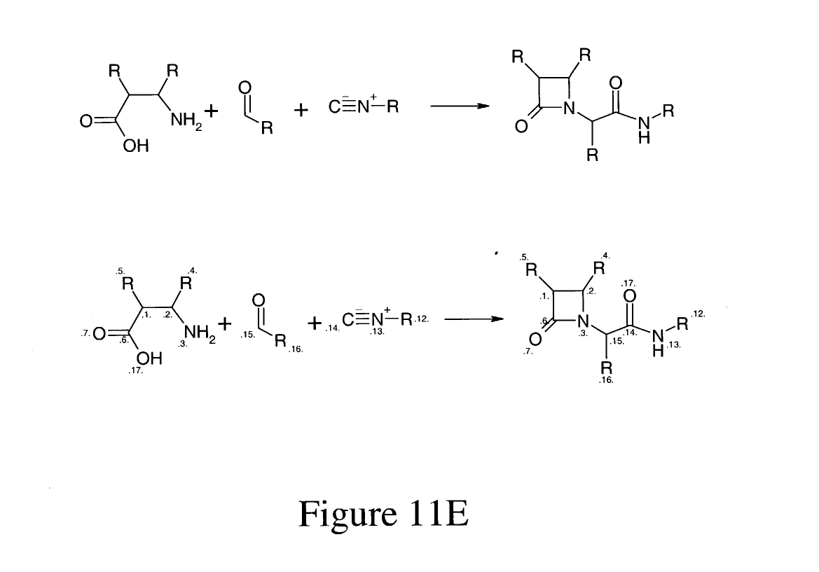

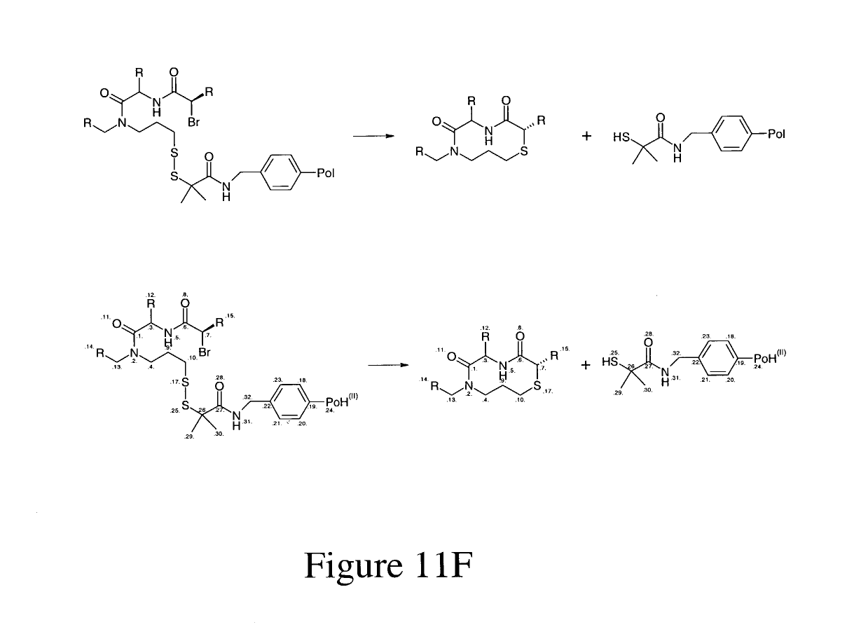

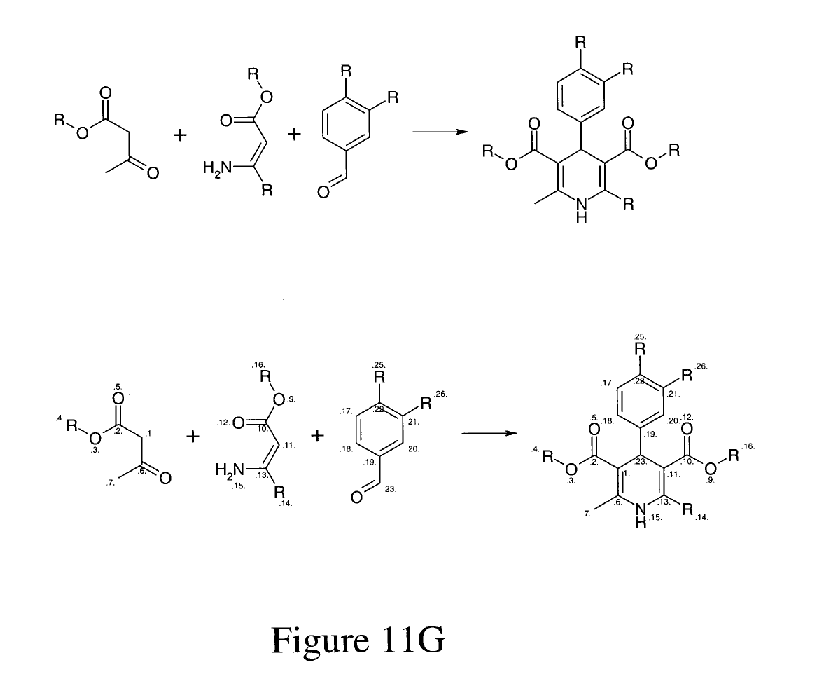

For the chemical reaction shown in FIG. 11A, the following SMARTS functions are generated.

Transform Function:

[A:20]([A:25](=[A:7])[A:10][A:4])([A,a;$([N;!H0;!H1][C;!H0](C(O[Pol])=O)[A,a]):3]([H])[H])[A,a:1].[A:21]([A:9 ][A:24](=[A:6])[A:11][A:8][A:22]1[a:26]2[a:16][a:12][a:14 ][a:18][a:28]2-[a:29]3[a:19][a:15][a:13][a:17] [a:27]13) ([A,a;$(C([OH1])(═O)[C;!HO]([N;!HO]C(O[C;!HO;!H1][C;!H0]1c2[c;! H0] [c;!H0][c;!H0][c; !H0]c2-c3 [c;!H0][c;!H0][c;!H0][c;!H0]c13)=O)[A,a]):23](=[A:5])[OH1])[A,a:2]>>[A:21]([A:9][A: 24](=[A:6])[A:11][A:8][A:22]1[a:26]2[a:16][a:12][a:14][a: 18][a:28]2-[a:29]3[a:19][a:15][a:13][a:17][a:27]13)([C:23](=[A:5])[N;!H0:3]([A:20]([A:25](=[A:7])[A:10][A:4])[A,a:1])[H])[A,a:2]

Processing Function:

[A:20]([A:25](=[A:7])[A:10][A:4])([N;!H0;!H1:3]([H]) [H])[A,a:1].[A:21]([A:9][A:24](=[A:6])[A:11][A:8][A:22]1[a:26]2[a:16][a:12][a:14][a:18][a:28]2-[a:29]3[a:19][a:15][a:13][a:17][a:27]13)([C:23](=[A:5])[OH1])[A,a:2]>>[A:21]([A:9][A:24](=[A:6])[A:11][A:8][A:22]1[a:26]2[a:16][a:12][a:14][a:18][a:28]2-[a:29]3[a:19][a:15][a:13][a:17][a:27]13 )([C:23](=[A:5])[N;!H0:3]([A:20]([A:25](=[A:7])[A:10][A:4 ])[A,a:1])[H])[A,a:2]

Matching components (with one reacting center atom appearing first):

C([OH1])(═O)[C;!H0]([N;!H0]C(O[C;!H0;!H1][C;!H0]1c2[c;!H0][c;!H0][c;!H0][c; !HO]c2-c3 [c;!H0][c;!H0][c; !H0][c;!H0]c13)=O)[A,a]

[N;!H0;!H1][C;!H0](C(O[Pol])=O)[A,a]

For the chemical reaction shown in FIG. 11B, the following SMARTS functions are generated.

Transform Function:

[A:12]([A,a;$([N+](#[C-])[C;!H0;!H1][A,a]):19]#[C-:11])[A,a:1].[A,a:2][A:13][A:21]1[A:17][A:20]([A,a;$([N;!H0;!H1][C@@H]([H])1[O][C@H]([H])([C;!H0;!H1][A,a])[C@@H]([H])([C@@H]([H])([C@H]([H])1[O][A,a])[O][A,a])[O][A,a]):8]([H])([H]))[A:22]([A:14][A,a:3])[A:24]([A:16][A,a:5])[A: 23]1[A:15][A,a:4].[A,a;$([C;!H0](═O)[A,a]):18](═O)([A,a:6 ])[H].[A:10]=[A,a;$(C([O;!H0])(═O)[A,a]):25]([O; !H0:9][H])[A,a:7]>>[A,a:1][A:12][N;!H0:19]([H])[C:11](=[O:9])[C@@H:18]([H])([A,a:6])[N:8]([A:20]1[A:17][A:21]([A:13][A,a:2])[A:23]([A:15][A,a:4])[A:24]([A:16][A,a:5])[A:22]1[A:14][A,a:3])[C:25](=[A:10])[A,a:7]

Processing Function:

[A:12]([N+]:19]#[C-:11])[A,a:1].[A,a:2][A:13][A:21]1[A:17][A:20]([N;!H0;!H1: 8]([H])([H]))[A:22]([A:14][A,a:3])[A:24]([A:16][A,a:5])[A:23]1[A:15][A,a:4].[C;!H0:18](═O)([A,a:6])[H].[A:10]=[C:25]([0;!H0:9][H])[A,a:7]>>[A,a:1][A:12][N;!H0:19]([H])[C:11](=[O:9])[C@@H:18]([H])([A,a:6])[N:8]([A:20]1[A:17][A:21]([A:13][A,a:2])[A:23]([A:15][A,a:4])[A:24]([A:16][A,a:5])[A:22]1[A:14][A,a:3])[C:25](=[A:10])[A,a:7]

Matching components (with one reacting center atom appearing first):

[N+](#[C-])[C;!H0;!H1][A,a]

[N;!H0;!H1][C@@H]([H])1[O][C@H]([H])([C; H0;!H1][A,a ])[C@@H]([H])([C@@H]([H])([C@H]([H])1[O][A,a])[O][A,a])[O][A,a]

[C; !H0](=)[A,a]

C([O;!H0])(═O)[A,a]

For the chemical reaction shown in FIG. 11C, the following SMARTS functions are generated.

Transform Function:

[A:6]([A,a;$([C;!H0](\[C;!H0;!H1][A,a])=[C;!H0]/C([C;!H0;!H1][A,a])=[C;!H0;!H1]):8](=[C;!H0:9]([C:12]([A:7][A,a:2])=[C;!H0;!H1:4]([H])[H])[H])[H])[A,a:1].[A:11](=[A:5 ])([A,a;$(C(C(═O)[A,a])(C#N)=NOS(═O)(═O)c1[cH1][cH1]c([CH 3])[cH1][cH1]1):13](C#N)=[N:10]OS(═O)(═O)c1[cH1][cH1]c([C H3])[cH1][cH1]1)[A,a:3]>>[A:7]([c:12]1[c;!H0:9]([c:8]([A: 6][A,a:1])[n:10][c:13]([A:11](=[A:5])[A,a:3])[c;!H0:4]1[H [H])[A,a:2]

Processing Function:

[A:6]([C;!H0:8](=[C;!H0:9]([C:12]([A:7][A,a:2])=[C;!H0;!H1:4]([H])[H])[H])[H])[A,a:1].[A:11](=[A:5])(C:13](C#N)=[N:10]OS(═O)(═O)c1[cH1][cH1]c([CH3])[cH1][cH1]1)[A,a:3]>>[A:7]([c:12]1[c;!H0:9]([c:8]([A:6][A,a:1])[n:10][c:13]([A:11](=[A:5])[A,a:3])[c;!H0:4]1[H])[H])[A,a:2]

Matching components (with one reacting center atom appearing first):

[C;!H0]([C;!H0;!H1][A,a])=[C;!H0]/C([C;!H0;!H1][A,a])=[C;!H0;!H1]

C(C(═O)[A,a])(C#N)=NOS(═O)(═O)c1[cH1][cH1]c([CH3])[cH1][cH1]1

For the chemical reaction shown in FIG. 11D, the following SMARTS functions are generated.

Transform Function:

[A,a:1][A:13][A:27]([A:24]([A,a:3])[A:31](=[A:8])[A:20][A:26]([A:15][A,a;$([S;!H0][C;!H0;!H1][C@H]([H])([N;!H0][C](=[O])[C;!H0]([N]([C;!H0;!H1][A,a])[C](=[O])[C@@H]([H])([A,a])[N]([C;!HO;!Hl][A,a])[C](=[O])[c]l[c;!H0][c]([c]([c; !H0][c; !H0]1)[N+](=[O])[O—])[F])[A,a])[C](═[O])[N]2[C;!H0;!H1][C;!H0;!H1][N]([C;!H0;!H1][C;!H0;!H1]2)[A,a]):7]([H]))[A:33](=[A:10])[A:30]1[A:18][A:16][A:29]([A,a:5])[A:17][A:19]1)[A:32](=[A:9])[A:25]([A,a:4])[A:28]([A:14][A, a:2])[A:34](=[A:11])[a:37]2[a:21][a:22][a:38]([A:35](=[A: 12])[A:6])[c:36]([F])[a:23]2>>[A:13]([A:27]1[A:24]([A:31](=[A:8])[A:20][A:26]([A:15][S:7][c:36]2[a:23][a:37]([A:34 ](=[A:11])[A:28]([A:14][A,a:2])[A:25]([A:32]1=[A:9])[A,a:4])[a:21][a:22][a:38]2[A:35](=[A:12])[A:6])[A:33](=[A:10])[A:30]3[A:18][A:16][A:29]([A:17][A:19]3)[A,a:5])[A,a:3]) [A,a:1]

Processing Function:

[A,a:1][A:13][A:27]([A:24]([A,a:3])[A:31](=[A:8])[A: 20][A:26]([A:15][S;!H0:7]([H]))[A:33](=[A:10])[A:30]1[A:18][A:16][A:29]([A,a:5])[A:17][A:19]1)[A:32](=[A:9])[A:25]([A,a:4])[A:28]([A:14][A,a:2])[A:34](=[A:11])[a:37]2[a:21][a:22][a:38]([A:35](=[A:12])[A:6])[c:36]([F])[a:23]2>>[A:13]([A:27]1[A:24]([A:31](=[A:8])[A:20][A:26]([A:15][S:7] [c:36]2[a:23][a:37]([A:34](=[A:11])[A:28]([A:14][A,a:2])[A:25]([A:32]1=[A:9])[A,a:4])[a:21][a:22][a:38]2[A:35](=[A:12])[A:6])[A:33](=[A:10])[A:30]3[A:18][A:16][A:29]([A:17 ][A:19]3)[A,a:5])[A,a:3])[A,a:1]

Matching components (with one reacting center atom appearing first):

[S;!H0][C;!H0;!H1][C@H]([H])([N;!H0][C](=[0])[C;!H0]([N]([C;!H0;!H1][A,a])[C](═[O])[C@H] ([H]) ([A,a]) [N] ([C;! H0;!H1][A,a])[C](═[O])[c]1[c;!H0][c]([c]([c;!H0][c;!H0]1) [N+](═[O])[O—])[F])[A, a])[C](═[O])[N]2[C; !H0;!H1][C; !H0;!H1][N]([C;!H0;!H1][C;!H0;!H1]2)[A,a]

For the chemical reaction shown in FIG. 11E, the following SMARTS functions are generated.

Transform Function:

[A,a;$([C;!H0](═O)[A,a]):9](═O)([A,a:1])[H].[C-:8]#[A,a;$([N+](#[C—])[A,a]):10][A,a:2].[A:11]([A:12]([C:13](=[A:7])[O;!H0:6][H])[A,a:4])([A,a;$([N;!H0;!H1][C;!H0]([C;!H0](C([O;!H0]) ═O)[A,a])[A,a]):5]([H])[H])[A,a:3]>>[A:11]1([A:12]([C:13](=[A:7])[N:5]1[C;!H0:9]([C:8](=[0:6])[N;!H0:10]([A,a:2])[H])([A,a:1])[H])[A,a:4])[A,a:3]

Processing Function:

[C;!H0:1])[H].[C-:8]#[N+]:10][A,a:2].[A:11]([A:12]([C:13](=[A:7])[O;!H0:6][H])[A,a:4])([N;!H0;!H1:5]([H])[H])[A,a:3]>>[A:11]1([A:12]([C:13](=[A:7])[N:5]1[C;!H0:9]([C:8](=[0:6])[N;!H0:10]([A,a:2])[H])([A,a:1])[H])[A,a:4])[A,a:3]

Matching components (with one reacting center atom appearing first):

[C;!H0](═O)[A,a]

[N+](#[C-])[A,a]

[N;!H0;!H1][C;!H0]([C;!H0](C([O;!H0])═O)[A,a])[A,a]

For the chemical reaction shown in FIG. 11F, the following SMARTS functions are generated.

Transform Function:

[A,a:1][A:10][A:25]([A:13][A:11][A:12][A,a;$([S]([C;!H0;!H1][C;!H0;!H1][C;!H0;!H1][N]([C;!H0;!H1][A,a])[C](=[O])[C;!H0]([A,a])[N;!H0][C](=[O])[C@@H]([H])([A,a])[Br])[S][C]([C;!H0;!H1;!H2])([C;!H0;!H1;!H2])[C](=[O])[N;!H0][C;!H0;!H1][c]1[c;!H0][c;!H0][c]([c;!H0][c;!H0]1)[Pol]):17][S:18][A:31]([A:4])([A:5])[A:27](=[A:8])[A:15][A:14][a:30 ]1[a:21][a:19][a:29]([A:6])[a:20][a:22]1)[A:28](=[A:9])[A:24]([A,a:3])[A:16][A:26](=[A:7])[C@@H:23]([H])([A,a:2])[Br]>>[A,a:1][A:10][A:25]1[A:13][A:11][A:12][S:17][C@@H:23]([H])([A,a:2])[A:26](=[A:7])[A:16][A:24]([A,a:3])[A:28]1=[A:9].[A:4][A:31]([A:5])([A:27](=[A:8])[A:15][A:14][a:30]1[a:21][a:19][a:29]([A:6])[a:20][a:22]1)[S;!H0:18][H]

Processing Function:

[A,a:1][A:10][A:25]([A:13][A:11][A:12][S:17][S:18][A:31]([A:4])([A:5])[A:27](=[A:8])[A:15][A:14][a:30]1[a:21][a:19][a:29]([A:6])[a:20][a:22]1)[A:28](=[A:9])[A:24]([A, a:3])[A:16][A:26](=[A:7])[C@@H:23]([H])([A,a:2])[Br]>>[A, a:1][A:10][A:25]1[A:13][A:11][A:12][S:17][C@@H:23]([H])([A,a:2])[A:26](=[A:7])[A:16][A:24]([A,a:3])[A:28]1=[A:9].[A:4][A:31]([A:5])([A:27](=[A:8])[A:15][A:14][a:30]1[a:21] [a:19][a:29]([A:6])[a:20][a:22]1)[S;!H0:18][H]

Matching components (with one reacting center atom appearing first):

[S]([C;!H0;!H1][C;!H0;!H1][C;!H0;!H1][N]([C;!H0;!H1][A,a])[C](═[O])[C;!H0]([A,a])[N;!H0][C](═[O])[C@@H]([H])([A,a])[Br])[S][C]([C;!H0;!H1;!H2])([C;!H0;!H1;!H2])[C](=[O])[N;!H0][C;!H0;!H1][c]1[c;!H0][c;!H0][c]([c;!H0][c;!H0]1)[Poll

For the chemical reaction shown in FIG. 11G, the following SMARTS functions are generated.

Transform Function:

[A:11]([A:20](=[A:8])[A,a;$([C;!H0;!H1](C(O[A,a])═O) C(═O)[C;!H0;!H1;!H2]):10]([C:19]([A:6])═O)([H])[H])[A,a:1 ].[A:12]([A:21](=[A:9])[A,a;$([C;!H0](C(O[A,a])═O)═C([N;!H0;!H1])[A,a]):14](=[A:18]([N;!H0;!H1:7]([H])[H])[A,a:3]) [H])[A,a:2].[A,a;$([C;!H0](═O)c1[c;!H0][c;!H0]c(c([c;!H0]1)[A,a])[A,a]):13](═O)([a:24]1[a:16][a:15][a:22]([A,a:4]) [a:23]([a:17]1)[A,a:5])[H]>>[A:12]([A:21](=[A:9])[C:14]1=[A:18]([N;!H0:7]([C:19]([A:6])=[C:10]([A:20](=[A:8])[A:11][A,a:1])[C;!H0:13]1([a:24]2[a:16][a:15][a:22]([A,a:4])[a:23]([A,a:5])[a:17]2)[H])[H])[A,a:3])[A,a:2]

Processing Function:

[A:11]([A:20](=[A:8])[C;!H0;!H1:10]([C:19]([A:6])═O) ([H])[H])[A,a:1].[A:12]([A:21](=[A:9])[C;!H0]:14](=[A:18]([N;!H0;!H1:7]([H])[H])[A,a:3])[H])[A,a:2].[C;!H0:13](═O) ([a:24]1[a:16][a:15][a:22]([A,a:4])[a:23]([a:17]1)[A,a:5])[H]>>[A:12]([A:21](=[A:9])[C:14]1=[A:18]([N;!H0:7]([C:19]([A:6])=[C:10]([A:20](=[A:8])[A:11][A,a:1])[C;!H0:13]1([a:24]2[a:16][a:15][a:22]([A,a:4])[a:23]([A,a:5])[a:17]2)[H])[H])[A,a:3])[A,a:2]

Matching components (with one reacting center atom appearing first):

[C;!H0;!H1](C(O[A,a])═O)C(═O)[C;!H0;!H1;!H2]

[C;!H0](C(O[A,a])═O)═C([N;!H0;!H1])[A,a][C;!H0](═O)c1[c;!H0][c;!H0]c(c([c;!H0]1)[A,a])[A,a]

Other Embodiments

While this invention has been described in terms of a few preferred embodiments, it should not be limited to the specifics presented above. Many variations on the above-described preferred embodiments may be employed. Therefore, the invention should be broadly interpreted with reference to the following claims.

What is claimed is:

1. A computer-implemented method for generating a reaction transform, the method comprising:
(a) receiving a representation of a chemical reaction for converting one or more generic reactants to one or more generic products, wherein the representation of the chemical reaction (i) maps atoms between the reactants and the products and (ii) specifies chemical properties for at least some atoms of the reactants and products;
(b) identifying reaction center atoms in the chemical reaction; and
(c) from said representation of the chemical reaction, creating a processing function for transforming representations of chemical building blocks matching said one or more generic reactants to corresponding representations of products, wherein the processing function comprises (i) representations of the reaction center atoms specifying one or more of said chemical properties and (ii) representations of non-reaction center atoms which do not include said one or more of said chemical properties.

2. The method of claim 1, wherein the non-reaction center atoms are characterized by mapping information between said reactants and products and by connectivity information specifying their connections with the reaction center atoms.

3. The method of claim 1, wherein at least some of the non-reaction center atoms are further characterized by an aliphatic or aromatic type, but are not characterized by a specific atom type.

4. The method of claim 1, further comprising:
from said representation of the chemical reaction, creating a matching function for identifying building blocks having substructures of one or more reactants of the chemical reaction, wherein the matching function comprises chemical properties for both reaction center atoms and non-reaction center atoms of the one more reactants.

5. The method of claim 4, further comprising:
creating the reaction transform from the matching function and the processing function; wherein the matching function and the processing function are syntactically separate in the reaction transform.

6. The method of claim 4, wherein the matching function comprises a general structural or property requirement of matching building blocks.

7. The method of claim 4, wherein the matching function comprises a conditional structural or property requirement of matching building blocks.

8. The method of claim 1, wherein (a) through (c) are performed by software to automatically generate the reaction transform.

9. The method of claim 1, further comprising automatically mapping an unmapped chemical reaction drawing to produce the representation of the chemical reaction received in (a).

10. The method of claim 1, wherein the representation of the chemical reaction comprises an electronic drawing of a chemical reaction.

11. The method of claim 1, wherein identifying reaction center atoms comprises identifying atoms having chemical bonds that are made, broken, or changed during the chemical reaction specified in the representation.

12. The method of claim 11, wherein identifying reaction center atoms further comprises identifying atoms having a change in charge, oxidation state, or chirality during the chemical reaction specified in the representation.

13. The method of claim 1, wherein the chemical properties comprise one or more of stereochemical configuration, charge, isotope, oxidation state, geometric configuration, hybridization, atom mass or isotope, van der Waals surface area, electron density, electronegativity, ionization energies, Lewis basicity or acidity, and hydrophobicity.

14. The method of claim 1, wherein the chemical properties comprise a stereochemical property.

15. The method of claim 1, wherein the processing function is represented in SMIRKS or SMARTS.

16. A computer program product comprising a machine readable medium on which is provided program instructions for generating a reaction transform, the program instructions comprising:
(a) code for receiving a representation of a chemical reaction for converting one or more generic reactants to one or more generic products, wherein the representation of the chemical reaction (i) maps atoms between the reactants and the products and (ii) specifies chemical properties for at least some atoms of the reactants and products;
(b) code for identifying reaction center atoms in the chemical reaction; and
(c) code for using said representation of the chemical reaction to create a processing function for transforming representations of chemical building blocks matching said one or more generic reactants to corresponding representations of products, wherein the processing function comprises (i) representations of the reaction center atoms specifying one or more of said chemical properties and (ii) representations of non-reaction center atoms which do not include said one or more of said chemical properties.

17. The computer program product of claim 16, wherein the code in (c) characterizes non-reaction center atoms by mapping information between said reactants and products and by connectivity information specifying their connections with the reaction center atoms.

18. The computer program product of claim 16, wherein the code in (c) further characterizes at least some of the non-reaction center atoms by an aliphatic or aromatic type, but not by a specific atom type.

19. The computer program product of claim 16, further comprising:
code for using said representation of the chemical reaction to create a matching function for identifying building blocks having substructures of one or more reactants of the chemical reaction, wherein the matching function comprises chemical properties for both reaction center atoms and non-reaction center atoms of the one more reactants.

20. The computer program product of claim 19, further comprising:
code for creating the reaction transform from the matching function and the processing function; wherein the matching function and the processing function are syntactically separate in the reaction transform.

21. The computer program product of claim 19, wherein the matching function comprises a general structural or property requirement of matching building blocks.

22. The computer program product of claim 19, wherein the matching function comprises a conditional structural or property requirement of matching building blocks.

23. The computer program product of claim 16, further comprising code for mapping an unmapped chemical reaction drawing to produce the representation of the chemical reaction received in (a).

24. The computer program product of claim 16, wherein the representation of the chemical reaction comprises an electronic drawing of a chemical reaction.

25. The computer program product of claim 16, wherein the code for identifying reaction center atoms comprises code for identifying atoms having chemical bonds that are made, broken, or changed during the chemical reaction specified in the representation.

26. The computer program product of claim 25, wherein the code for identifying reaction center atoms further comprises code for identifying atoms having a change in charge, oxidation state, or chirality during the chemical reaction specified in the representation.

27. The computer program product of claim 16, wherein the chemical properties comprise one or more of stereochemical configuration, charge, isotope, oxidation state, geometric configuration, hybridization, atom mass or isotope, van der Waals surface area, electron density, electronegativity, ionization energies, Lewis basicity or acidity, and hydrophobicity.

28 The computer program product of claim 16, wherein the chemical properties comprise a stereochemical property.

29. The computer program product of claim 16, wherein the processing function is represented in SMIRKS.

30. The computer program product of claim 16, wherein the processing function contains information about the intra-molecular character of the chemical reaction.

31. A computer-implemented method of generating a virtual product of a chemical reaction from a set of virtual building blocks, the method comprising:
(a) providing a chemical transform specifying a conversion of one or more generic reactants to one or more generic products, wherein the chemical transform comprises a matching function for matching building blocks and a syntactically separate processing function for mapping matched building blocks to products;
(b) using said matching function to select a building block from said set of virtual building blocks and identify one or more reacting atoms in the selected building block;
(c) using the processing function to modify one or more chemical properties or bond connections of the reacting atoms; and
(d) generating a virtual product comprising non-reacting features from the selected building block and modified reacting atoms created at (c)

32. The method of claim 31, further comprising repeating (b) through (e) multiple times to create a library of virtual products.

33. The method of claim 31, wherein (a) through (e) are automatically performed by software.

34. The method of claim 31, wherein the matching function comprises a substructure representing a generic reactant.

35. The method of claim 31, wherein using said matching function to identify reacting atoms comprises comparing atoms of the building block to reaction center atoms specified by the matching function.

36. The method of claim 31, wherein the processing function comprises a representation of the conversion of one or more generic reactants to one or more generic products, in which non-reaction center atoms are represented without chemical properties, aside from mapping and connectivity information, while reaction center atoms are represented with reaction center atoms.

37. The method of claim 31, wherein the chemical properties comprise one or more of stereochemical configuration, charge, isotope, oxidation state, geometric configuration, hybridization, atom mass or isotope, van der Waals surface area, electron density, electronegativity, ionization energies, Lewis basicity or acidity, and hydrophobicity.

38. A computer program product comprising a machine readable medium on which is provided program instructions for generating a virtual product of a chemical reaction from a set of virtual building blocks, the program instructions comprising:
(a) code for providing a chemical transform specifying a conversion of one or more generic reactants to one or more generic products, wherein the chemical transform comprises a matching function for matching building blocks and a syntactically separate processing function for mapping matched building blocks to products;
(b) code for using said matching function to select a building block from said set of virtual building blocks and identify one or more reacting atoms in the selected building block;
(c) code for using the processing function to modify one or more chemical properties or bond connections of the reacting atoms; and
(d) code for generating a virtual product comprising non-reacting features from the selected building block and modified reacting atoms created at (c).

39. The computer program product of claim 38, wherein the program instructions further comprise code for repeating (b) through (e) multiple times to create a library of virtual products.

40. The computer program product of claim 38, wherein the matching function comprises a substructure representing a generic reactant.

41. The computer program product of claim 38, wherein the code for using said matching function to identify reacting atoms comprises code for comparing atoms of the building block to reaction center atoms specified by the matching function.

42. The computer program product of claim 38, wherein the processing function comprises a representation of the conversion of one or more generic reactants to one or more generic products, in which non-reaction center atoms are represented without chemical properties, aside from mapping and connectivity information, while reaction center atoms are represented with reaction center atoms.

43. The computer program product of claim 38, wherein the chemical properties comprise one or more of stereochemical configuration, charge, isotope, oxidation state, geometric configuration, hybridization, atom mass or isotope, van der Waals surface area, electron density, electronegativity, ionization energies, Lewis basicity or acidity, and hydrophobicity.
